SEQUENCE LISTING (27 sequences)

SEQ ID NO: 1 - protein, 4 aa, synthetic construct
  REGION 1..4: note = ARTIFICIAL SEQUENCE
KTFK

SEQ ID NO: 2 - protein, 4 aa, synthetic construct
  REGION 1..4: note = synthetic peptide
GQPR

SEQ ID NO: 3 - protein, 4 aa, synthetic construct
  REGION 1..4: note = synthetic peptide
RSRK

SEQ ID NO: 4 - protein, 4 aa, synthetic construct
  REGION 1..4: note = Synthetic peptide
KTAK

SEQ ID NO: 5 - protein, 4 aa, synthetic construct
  REGION 1..4: note = Synthetic peptide
KAYK

SEQ ID NO: 6 - protein, 4 aa, synthetic construct
  REGION 1..4: note = Synthetic peptide
KFYK

SEQ ID NO: 7 - protein, 4 aa, synthetic construct
  REGION 1..4: note = Artificial sequence
TKPR

SEQ ID NO: 8 - protein, 5 aa, synthetic construct
  REGION 1..5: note = synthetic peptide
KTTKS

SEQ ID NO: 9 - protein, 5 aa, synthetic construct
  REGION 1..5: note = synthetic peptide
KTSKS

SEQ ID NO: 10 - protein, 6 aa, synthetic construct
  REGION 1..6: note = Synthetic peptide
GKTTKS

SEQ ID NO: 11 - protein, 6 aa, synthetic construct
  REGION 1..6: note = synthetic peptide
VGVAPG

SEQ ID NO: 12 - protein, 4 aa, synthetic construct
  REGION 1..4: note = Synthetic peptide
  MOD_RES 1: note = amidation by a Elaidoyl chain on the N terminal end
KTAK

SEQ ID NO: 13 - protein, 4 aa, synthetic construct
  REGION 1..4: note = Synthetic peptide
  MOD_RES 1: note = amidation by a Elaidoyl chain on the N terminal end
KAYK

SEQ ID NO: 14 - protein, 4 aa, synthetic construct
  REGION 1..4: note = Synthetic peptide
  MOD_RES 1: note = amidation by a Elaidoyl chain on the N terminal end
KFYK

SEQ ID NO: 15 - protein, 4 aa, synthetic construct
  REGION 1..4: note = synthetic peptide
  MOD_RES 1: note = amidation by a Palmitoyl chain on the N terminal end
GQPR

SEQ ID NO: 16 - protein, 4 aa, synthetic construct
  REGION 1..4: note = ARTIFICIAL SEQUENCE
  MOD_RES 1: note = amidation by a Palmitoyl chain on the N terminal end
KTFK

SEQ ID NO: 17 - protein, 5 aa, synthetic construct
  REGION 1..5: note = synthetic peptide
  MOD_RES 1: note = amidation by a Palmitoyl chain on the N terminal end
KTTKS

SEQ ID NO: 18 - protein, 5 aa, synthetic construct
  REGION 1..5: note = synthetic peptide
  MOD_RES 1: note = amidation by a Palmitoyl chain on the N terminal end
KTSKS

SEQ ID NO: 19 - protein, 5 aa, synthetic construct
  REGION 1..5: note = synthetic peptide
  MOD_RES 1: note = amidation by a Palmitoyl chain on the N terminal end
  VAR_SEQ 5: note = MISC_FEATURE - Xaa being either a Proline P or a Leucine L
YGGFX

SEQ ID NO: 20 - protein, 6 aa, synthetic construct
  REGION 1..6: note = Synthetic peptide
  MOD_RES 1: note = amidation by a Palmitoyl chain on the N terminal end
  VAR_SEQ 6: note = MISC_FEATURE - Xaa being Trp, Phe, Tyr, Tic, 7-hydroxy-Tic or Tpi
HLDIIX

SEQ ID NO: 21 - protein, 6 aa, synthetic construct
  REGION 1..6: note = synthetic peptide
  MOD_RES 1: note = amidation by a Palmitoyl chain on the N terminal end
GKTTKS

SEQ ID NO: 22 - protein, 6 aa, synthetic construct
  REGION 1..6: note = synthetic peptide
  MOD_RES 1: note = amidation by a Palmitoyl chain on the N terminal end
VGVAPG

SEQ ID NO: 23 - protein, 6 aa, synthetic construct
  REGION 1..6: note = synthetic peptide
  MOD_RES 1: note = Acetylation on the N-terminal end
EEMQRR

SEQ ID NO: 24 - protein, 5 aa, synthetic construct
  REGION 1..5: note = synthetic peptide
YAGFL

SEQ ID NO: 25 - protein, 4 aa, synthetic construct
  REGION 1..4: note = synthetic peptide
AHSH

SEQ ID NO: 26 - protein, 6 aa, synthetic construct
  REGION 1..6: note = synthetic peptide
SIKVAV

SEQ ID NO: 27 - protein, 6 aa, synthetic construct
  REGION 1..6: note = synthetic peptide
GPQGPQ

(19) United States
(12) Patent Application Publication
(10) Pub. No.: US 2026/0207470 A1
(43) Pub. Date: Jul. 23, 2026

(54) Method For Obtaining An Extract Of Plant Origin, A Composition Containing It And Its Cosmetic Use

(71) Applicant: Sederma, Le Perray en Yvelines (FR)

(72) Inventors: Céline BONDIL, Le Perray en Yvelines (FR); Philippe MONDON, Le Perray en Yvelines (FR); Frédéric DE BAENE, Le Perray en Yvelines (FR); Caroline RINGENBACH, Le Perray en Yvelines (FR); Alex SAUNOIS, Le Perray en Yvelines (FR)

(73) Assignee: Sederma, Le Perray en Yvelines (FR)

(21) Appl. No.: 19/139,740

(22) PCT Filed: Dec. 21, 2023
(86) PCT No.: PCT/EP2023/087438

§ 371 Date: Jun. 16, 2025

(30) Foreign Application Priority Data
Dec. 22, 2022 (FR) FR2214243

(51) Int. Cl. A61K 8/9789; A61Q 19/00; C12N 5/04

(52) U.S. Cl. CPC A61K 8/9789; A61Q 19/00; C12N 5/04

(57) ABSTRACT

The present invention provides a method for obtaining an extract of plant origin by in vitro culture from a line of undifferentiated or dedifferentiated plant cells, comprising the addition of at least one aglycone flavonoid to the culture medium. The extract obtained contains a set of secondary metabolites of interest. It can be used advantageously for non-therapeutic cosmetic treatment of the skin and appendages.

### CROSS REFERENCE TO RELATED APPLICATIONS

This is the U.S. National Phase application of PCT/EP2023/087438, filed Dec. 21, 2023, which claims priority to French Patent Application No. FR2214243, fled Dec. 22, 2022, the disclosures of each of these applications being incorporated heroin by reference in their entireties for all purposes.

### REFERENCE TO AN ELECTRONIC SEQUENCE LISTING

The contents of the electronic sequence listing (DIAN_FR ST26.xml; Size: 30 KB; and Date of Creation: Dec. 21, 2023) is herein incorporated by reference in its entirety.

### FIELD OF THE INVENTION

The present invention relates to a new method for obtaining an extract of plant origin, a composition containing it and its cosmetic use.

### BACKGROUND OF THE INVENTION

The present invention relates in particular to ingredients and compositions for the cosmetics, cosmeceutical, dermopharmacy, and hygiene and personal care products industry, the extract of plant origin being used for the treatment of the skin of mammals, humans or animals, and its appendages.

An extract of plant origin can be obtained in the conventional manner directly from a plant or by in vitro culture of plant calls, tissues, or plant organs.

The present invention relates more particularly to a manufacturing method by in voice culture.

Obtaining extracts of plant origin by in vitro culture has numerous advantages on the ago-industrial route (cultivation of plants in open field and subsequent extraction industrially). Because of the total control of the culture conditions, the extracts obtained by in vitro culture are free of toxic substances (herbicides, pesticides, fertilizers, heavy metals, and other contaminants, such as those which may originate from plant parasites). Moreover, strict control of the in vitro culture conditions reduces the risk of spontaneous variation of the strain and guarantees a reproducible profile of secondary metabolites which correspond to the molecules of interest sought, unlike culturing in open fields where the problem of variability arises, linked to the climatic, meteorological, and geographical conditions and their hazards. Furthermore, this technology overcomes obstacles such as the natural biological cycle of the plant and the seasonality of production of secondary metabolites, allowing a supply that is safer and more rapid. Moreover, the environmental impact is minimal because it substantially limits the consumption of water, avoids exploitation of arable soil, and prevents soil pollution. In addition, biodiversity is preserved since a plant or even a seed is sufficient to initiate a new in vitro culture. Finally, this technology offers the possibility of carrying out controlled and relatively rapid protocols for increasing the yields of certain molecules, especially those produced in small quantities in the plant for example by using the elicitation of the in vitro cultures).

Among the existing techniques of in vitro culture of plants, the invention aims more particularly at the culture of undifferentiated or de differentiated cels: this method comprises first the creation of highly proliferative cell lines in agar medium either from meristematic cells which are undifferentiated cells, or from dedifferentiated cells which grow in the form of healing masses called calluses, following the removal and cutting of a plant fragment (also called explant), of plant, leaf, stem, root or other. Thereafter this cell line is cultured, first on agar medium, then in liquid medium so as to substantially increase the biomass. During the growth cycle and under medium conditions to be defined and optimized, secondary metabolites will be synthetized by the plant calls of the biomass which will constitute the molecules of cosmetic interest. The culture is then stopped and subjected to extraction at the optimum time to obtain a maximum quantity of molecules of interest. It is also possible to use as starting material existing cell lines that are already commercially available.

Thus, the method of the invention comprises schematically:
optionally, in a first time, a step of creation and selection of a cell line capable of producing cellular biomass on a large-scale according to pre established criteria (constant phenotype and optimal and constant production of the chosen metabolites, capacity to proliferate);
then, in a second time, a pre-culture step from this selected line for increasing the number of cels in a biomass;
then, in a third time, a culture step in a bioreactor to proliferate the cellular biomass, optionally with an elicitation step:
then, in a fourth time, a treatment step of treating the obtained cellular biomass to recover said cellular extract of plant origin according to the invention which will include at least the expected secondary metabolites of cosmetic interest.

Until today, the described in vitro techniques allow to produce, compared to classic extracts, original extracts in that they contained caffeic acid derivatives as secondary metabolites at interesting concentrations to provide a cosmetic activity. The following patents and applications can be cited.

EP1736167 describes the obtaining of an extract of plant origin from plant cels of Syringa vulgaris comprising an advantageous isoverbascoside quantity which belongs to the phenylpropanoid glycoside family.

EP2319914 describes the in vitro obtaining for a theoretical list of plant species of an extract comprising phenylpropanoid glycosides or caffeoylquinic acids which are derivatives of caffeic acid.

WO2016/113659 describes the obtaining of an extract of plant origin from undifferentiated or dedifferentiated plant cells of Leontopodium alpinium comprising an advantageous phenylpropanoid glycosides quantity including leontopodic acids A and B.

WO2017/163174 describes the obtaining of an extract of plant origin from undifferentiated or dedifferentiated plant calls of Leontopodium alpinium comprising an advantageous phenylpropanoid glycosides quantity including leontopodic acids A and B.

WO2020/165365 describes the obtaining of an extract of plant origin from undifferentiated or dedifferentiated plant calls of Buddleja davidii Franch, comprising an advantageous phenylpropanoid glycosides quantity including verbascoside.

To improve the biological activity and the originality of the extracts, it is often proposed to stimulate the plant cells production of secondary metabolites existing only in very small quantities in the biomass by an elicitation, for example chemical or physical.

### SUMMARY OF THE INVENTION

The aim of the present invention is to propose a new manufacturing method by in vitro plant cell culture which makes it possible to obtain an extract comprising other secondary metabolites than those described in the state of the art, said extract exhibiting a biological activity, especially cosmetic. According to a first object, the present invention proposes a method for obtaining an extract of plant origin by in vitro plant culture, comprising, from a line of undifferentiated or dedifferentiated plant cell, the following steps successively:
a pre-culture stop, intended to amplify the biomass of said plant cells;
a culture step of the biomass in a bioreactor comprising at least one proliferation phase; and
a treatment step of the harvested biomass to produce said extract comprising secondary metabolites of interest;
   wherein at the culture step in the bioreactor at least one aglycon flavonoid is added to the culture medium.

### BRIEF DESCRIPTION OF THE DRAWINGS

The present invention will be better understood in the light of the following description of embodiments, studies and figures described below.

FIG. 1 represents a chromatogram monitoring the secondary metabolites produced by a call biomass of Monarda didyma just before addition of naringenin.

FIG. 2 represents a chromatogram of biomass monitoring as in FIG. 1 but seven days after the addition of naringenin, illustrating the bioconversion of naringenin into a set of glycosylated derivatives thereof.

FIG. 3 represents a monitoring chromatogram as in FIG. 1 for a cell biomass of Lavandula minutes after addition of luteolin.

FIG. 4 represents a chromatogram monitoring the biomass as in FIG. 3 but eight days after the addition of luteolin, illustrating the bioconversion of luteolin into a set of glycosylated derivatives thereof.

FIG. 5 represents a graph illustrating the kinetics of production of glycosylated flavonoids and rosmarinic acid during a cell culture of Monarda didyma according to the method of the invention.

FIG. 6 represents a graph of deformation of the skin under the effect of mechanical deformation as a function of time.

### DETAILED DESCRIPTION OF THE INVENTION

Advantageously, a new extract enriched in secondary metabolites of a new class is obtained according to the method of the invention. At the end of the method, a plant extract which includes in its secondary metabolites a set of glycosylated flavonoids is recovered. By comparison, a plant extract obtained according to the method of the prior art does not contain this type of secondary metabolites.

Surprisingly, the plant cells were able to integrate the aglycone flavonoid which had been added to the culture medium and to metabolize it. The aglycone flavonoid acted as a precursor.

“Aglycone” commonly means without any osidic radical.

Several mechanisms could explain the surprising effect obtained according to the invention.

The aglycone flavonoid can revealed particular metabolic pathways which were indeed present but which were not yet used due to lack of precursors. It is by revealing and stimulating these metabolic pathways that aglycone flavonoids were converted into glycosylated flavonoids.

Aglycone flavonoids can also played the role of epigenetic probe capable of sending signals to the DNA of calls, thus making it possible to generate and/or mobilize certain particular enzymes. These enzymes, responsible for cell differentiation, then induced the opening of certain specific metabolic pathways.

Finally, aglycone flavonoid con acted as epigenetic probe capable of unlocking certain DNA reading areas. These mechanisms may have taken place alone or in combination, advantageously making it possible to offer a cumulative or synergistic effect to produce a particularly interesting composition of molecules of interest. Glycosylated flavonoids are of interest in cosmetics, in that they can improve or beautify the general condition of the skin and its appendages. They are known for their particularly antioxidant properties. The detailed description given below presents examples of extracts obtained according to the method of the invention, having very interesting cosmetic properties supported by in vitro and in vivo tests.

Preferably according to the invention, the aglycone flavonoid added to the biomass is chosen from at least one aglycons flavone and/or one aglycone flavanone.

Preferably, the at least one flavanone aglycone is chosen from naringenin, eriodictyol and butin, or a mixture thereof, and the at least one flavone aglycone comprises luteolin and apigenin, or a mixture thereof. More preferably according to the invention, the aglycone flavonoid added to the biomass is naringenin and/or luteolin. It is these two flavonoids, each belonging to a different class, that are exemplified in the detailed description. Naringenin is also known as naraginin, naringinin, naringetol or 5,7-dihydroxy-2-(4-hydroxyphenyl)chroman-4-one (C₁₅H₁₂O₅) and has the structural formula:

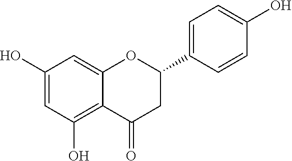 [SMILES: O=C1C[C@@H](c2ccc(O)cc2)Oc2cc(O)cc(O)c21]

Luteolin is also known as luteolol or 6,7-dihydroxy-2-(3,4-dihydroxyphenyl)-chromen-4-one (C₁₅H₁₀O₅) and has the structural formula:

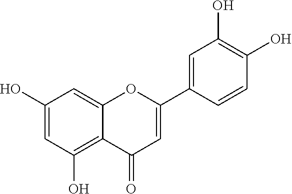 [SMILES: O=c1cc(-c2ccc(O)c(O)c2)oc2cc(O)cc(O)c12]

The choice of one or more aglycone flavonoids can be made depending on the types of glycosylated molecules derived from these flavonoids that are being sought.

The quantity of the one or more aglycone flavonoids to be added is determined according to what the plant cells can accept and exploit, firstly to ensure rapid growth of the biomass and secondly to enable maximum yield of secondary metabolite synthesis, all without having any toxic effect on the cells.

The addition of the aglycone flavonoid is carried out at the bioreactor culture step, preferably during an exponential phase of cell growth, more preferably during the exponential phase of the last proliferation cycle.

By “cycle” is meant the period between two additions of fresh culture medium to the bioreactor or between the addition of fresh culture medium and the recovery of the cells (last cycle). A cycle can be repeated several times. It is used to make the cells proliferate. Each cycle takes place in several phases determined by their proliferation rates. It is during the exponential phase that the calls are the most actives and therefore the most capable of metabolizing the aglycone flavonoids added to the culture medium into a set of glycosylated derivatives of these flavonoids.

According to another feature of the invention, an optional elicitation step can be carried out at the bioreactor culture step, preferably also during an exponential phase of cell growth, more preferably during the exponential phase of the last proliferation cycle, still more preferably simultaneously with the addition of the aglycone flavonoid.

The elicitor quantity to add is selected as for the aglycone flavonoid depending on what the cells can accept and exploit.

Generally, the elicitation of interest compounds can be performed by the addition to the culture of microbial fractions (in particular Sta as yeasts); the addition to the molecules culture of biological origin such as for example chitosan, methyl jasmonate, jasmonic acid and salicylic acid: the addition to the molecules culture of non-biological origin such as, for example, paclobutrazol; the application to the culture of a variation in temperature, pH or an osmotic stress induced by a non-metabolizable sugar, such as for example mannitol; to the use of an even more drastic depletion in macroelements and sugar of the medium; the addition to the culture of adsorbent resins stich, in addition to eliciting the production of the compounds of interest, con trap them.

Preferably according to the invention, the added elicitor is the methyl jasmonate.

Surprisingly, the Applicant has noticed that the addition of methyl jasmonate combined with the addition of at least one aglycone flavonoid not only makes it possible to increase the concentration of secondary metabolites which is only present in very small quantity naturally in the cells, such as rosmarinic acid, but also allows to further increase the concentration of glycosylated flavonoids (see results of the analyses detailed below).

According to other characteristics of the method of the invention, the biomass treatment step for recovering the extract according to the invention after the culture step comprises a conventional separation step to eliminate the culture medium (the supernatant) and recover the cellular biomass. This step can be done for example by filtration or centrifugation. The recovered biomass consists of partially lysed or whole calls, in the form of clusters or individual cells. The secondary metabolites of interest, including all glycosylated flavonoids, are found in the intracellular content of plant calls of the recovered biomass.

The cellular extract according to the invention is thus obtained which contains the whole cells or partially lysed calls, more precisely an extract which contains both the intracellular content, including the secondary metabolites of interest, and the cell walls and/or debris. This extract can be used for manufacturing a cosmetic composition, optionally after having undergone additional treatment to break up the cel aggregates, in particular by homogenization under high pressure or any other appropriate technique. The composition according to the invention can be more or less concentrated in cellular extract, thus forming either a cosmetic ingredient, or a final cosmetic formulation intended for the final consumer which can be prepared from this cosmetic ingredient. Optionally, the treatment step can include a following stop of releasing the cellular contents outside the plant calls which can be carried out according to the different known processes which can be combined: under heating, by maceration, grinding, decoction, infusion, pressure, leaching, diffusion, distillation, liquid/liquid separation, with ultrasound, microwaves, or by lysing the calls by any appropriate chemical or physical process. It is also possible to extract the biomass with a supercritical or subcritical fluid.

According to the invention, the release of the intracellular contents containing the secondary metabolites of interest is carried out preferentially by osmotic diffusion or by call lysis.

Osmotic diffusion is carried out by adding a solvent poor in water to the biomass. Preferably according to the invention, butylene glycol, propanediol, propylene glycol, pentylene glycol, glycerol or a mixture of these are added. More preferably, a glycerol is added.

An extract comprising the intracellular content and cellular debris is thus obtained. This extract can be used as is to manufacture a cosmetic composition.

Still optionally, the treatment step can include a subsequent step to eliminate the cellular debris from the obtained extract, for example by any type of filtration of centrifugation. Thanks to this step a purified and clear extract is advantageously obtained which can be used as an active ingredient in cosmetic compositions. The extract is more transparent and has advantageous formulation qualities, such as the possibility of helping the preparation of gel or serum type products.

According to another particular characteristic of the method of the invention, the plant cell lines used in the pre-culture step is preferably prepared extemporaneously.

Creation of a cell line includes the phases: 1) induction of calluses (clusters of undifferentiated or dedifferentiated cells), 2) selection of the best calluses and 3) optimization on the calluses selected in a culture medium.

The culture medium, liquid or agar, includes macroelements, microelements, hormones, vitamins and sugars. It is selected to promote the growth and production of interest molecules, namely glycosylated derivatives of the aglycone flavonoids added at the culture stage.

More specifically:
1) Callus induction is carried out in agar culture medium and can be performed with all parts of the plant, including leaf, fruit, root bud, seed stem, branch, meristematic tissue and cambium. Preferably, according to the invention, the culture of plant calls is performed from the leaves of the plant.
2) The selection of the best lines, before transfer to a liquid base medium, is carried out according to the invention according in particular to the following criteria: strong proliferative capacity, tender and friable texture, homogeneous colour, good dispersion in liquid medium and stability in the time of these parameters.
3) Optimization of the characteristics of growth and production of secondary metabolites in liquid medium for the selected Ines involves the choice of the most suitable media, firstly to ensure rapid growth of the biomass and secondly to allow maximum synthesis yield of primary and secondary metabolites by the cels at the end of the exponential phase (stopping of multiplication) in order to facilitate its passage into a bioreactor.

A cell line thus prepared can be used directly or preserved for later use.

According to the method of the invention, the culture medium, liquid or agar, used in the different stages classically comprises macroelements, microelements, hormones, vitamins and sugars. It is selected to promote the growth and production of molecules of interest, namely according to the invention of glycosylated flavonoids. The culture medium can also be selected to allow the growth of other secondary metabolites by the plant calls, in particular those naturally present in the cell Ine but in very small quantities, such as for example classic caffeic acids, in particular rosmarinic acid. as shown in the examples given below. The extract obtained will advantageously include a whole set of secondary metabolites that can ac synergistically and confer to the extract original and now properties in cosmetics.

The method of the invention can be implemented starting from a line of any plant.

The plant can be selected from the families of plants known to produce contracts of interest in cosmetics, in particular from the Lamiaceae, Boraginaceae, Nyctaginaceae, Asteraceae or Apiaceae family, preferably the Lamiaceae and/or Nyctaginaceae families.

More preferably, the plant can be chosen from the following genus: Abronia, Acinos, Acleisanthes, Ajuga, Allionia, Andradea, Anubcautis, Ballota, Belerria, Boerhavia, Bougainvillea, Catemintha, Caribea, Clinopodium, Cotignonia, Commicarpus, Cyphomeris, Galeopsis, Glechoma, Guapira, Hyssopus, Lamium, Lavandula, Leucaster, Marrubium, Melisssa, Melttis, Mentha, Mirabilis, Monarda, Neea, Nepeta, Nyctaginia, Ocimum, Okenia, Origanum, Orthosiphon, Perilla, Phaeoptilum, Pisonia, Pisoniella, Phlomis, Plectranthus, Prunella, Ramisia, Reichenbachia, Rosmarinus, Salvia, Satureja, Stachys, Teucrium, Thymus, Tripterocalyx and Salpianthus.

More preferably according to the invention, the plant is selected from the following genus: Abronia, Allionia, Anulocautis, Bougainvillea, Lavandula, Marrubium, Melissa, Mentha, Marabils, Monarda, Ocimum, Ogiranum, Orthosiphon, Perilla, Pisonia, Pisoniella, Prunella, Rosmarinus, Salvia, Satureja and Thymus.

More preferably, the plant belongs to the genus Abronia, Monarda and/or Lavandula.

The genus Monarda belongs to the Lamiaceae family and includes several species, including those known: Monarda austroappatachiana, Monarda bartlettii, Monarda bradburiana, Monarda brewis, Monarda citriodora, Monarda clinopodia, Monarda clinopodioides, Monarda didyma, Monarda eplingiana, Monarda fistulosa, Monarda fruticulosa, Monarda humillis, Monarda lindheimeri, Monarda tureola, Monarda maritima, Monarda media, Monarda pectinata, Monarda pringlei, Monarda punctata, Monarda russeliana, Monarda stanfieldii, Monarda viridissima.

The Applicant was particularly interested in the Monarda didyma species.

Monarda didyma is a honey, nectar, aromatic and edible plant native to eastern North America. Monarda, originally described by Nicolas Monardes, is also known as Oswego ten, but also Melisse d′Or″ or Bee Balm. Native Americans used macerations of leaves in oil for hair care or for their antiseptic power on pimples, or to combat colds.

The Lavandula genus belongs to the Lamiaceae family and includes several species, including those known: Lavandula×alportelensis, Lavandula angustifolia, Lavandula antineae, Lavandula aristibracteata, Lavandula atriplicifolia, Lavandula austroapennina, Lavandula pibinnata, Lavandula bramwellii, Lavandula buchii, Lavandula Lavandula dintata, Lavandula dhofarensis, Lavandula erythraeae, Lavandula galgalloensis, Lavandula gibsonii, Lavandula×ginginsii, Lavandula hasikensis, Lavandula×heterophylla, Lavandula×intermedia, Lavandula lanata, Lavandula latifolia, Lavandula×limae, Lavandula×losae, Lavandula macra, Lavandula mairei, Lavandula maroccana, Lavandula minutolii, Lavandula multifida, Lavandula nimmoi Benth, Lavandula nooruddinii, Lavandula pedunculata, Lavandula pinnata, Lavandula pubescens, Lavandula quishnensis, Lavandula rejdalii, Lavandula rotundifolia, Lavandula saharica, Lavandula samhanensis, Lavandula setifera, Lavandula somaliensis, Lavandula stoechas, Lavandula sublepidota, Lavandula subruda, Lavandula tenuisects, Lavandula vinidis.

The Applicant was particularly interested in the Lavandula angustifolia species.

Lavandula angustifolia is a plant appreciated for its smell. It is also known as lavender, true lavender of narrow leaved lavender. The flower is generally used to obtain essential oil with multiple virtues such as anti-inflammatory, antiseptic, healing and antibacterial.

The Abronia genus belongs to the Nyctaginaceae family and includes several species, including those known: Abronia alba, Abronia alpina, Abronia ameliae, Abronia ammophila, Abronia angustifolia, Abronia argillosa, Abronia bigelovii, Abronia carletonii, Abronia elliptics, Abronia fragans, Abronia gracilis, Abronia insulanis, Abronia latifolia, Abronia macrocarpa, Abronia maritima, Abronia mellifera, Abronia minor, Abronia nana, Abronia neurophylla, Abronia platyphylla, Abronia pogonantha, Abronia turbinata, Abronia umbellate, and Abronia villosa

The Applicant was particularly interested in the Almunia villosa species.

Abronia villosa is a species of sand-verbena known by the common names: desert sand verbena and chaparral sand verbena. The Paiute Native Americans used this plant as a diuretic. The Shoshone used the crushed root as a poultice for burns.

According to other more particular features of the method of the invention:

Concerning the pre-culture step, the selected cel line is multiplied to obtain a sufficient amount of undifferentiated or dedifferentiated cell biomass in order to carry out the large-scale production step, i.e., the culture step in a bioreactor.

The following sub-steps are implemented:
a) Inoculation of the selected line in a liquid culture medium and culture for a sufficient time to obtain a quantity of biomass multiplied by 2 or even 3 times compared to the original biomass. This first step is carried out in a container with a capacity of between 0.5 L and 2 L.
b) Optionally, transfer of all or part of the suspension obtained in a into a fresh liquid culture medium and culture again for a sufficient time to obtain a biomass quantity multiplied by 2 or even 3 times compared to the quantity of biomass at the start of the cycle.

This step b) constitutes a cycle, which can be repeated several times.
c) Optionally, repetition of step b).

Concerning the bioreactor culture step, the best call lines stabilized at the pre-culture stop are transferred into the bioreactor containing a liquid culture medium for cell proliferation Steps b) and c) carried out for the pre-culture are repeated in a container having a volume adapted to the biomass.

It is during the exponential proliferation phase of the last cycle, i.e., approximately between 7 and 12 days after the last initiation of the culture in the bioreactor, that the addition of the one or more aglycon flavonoids is preferentially carried out.

The culture is stopped when the desired level of secondary metabolites is reached, preferably approximately between 2 to 7 days after the addition of the one or more aglycone flavonoids. To reach this level, where necessary, as seen above, a culture medium is used.

According to the method of the invention, this culture medium, liquid or agar, used in the pre-culture or culture steps classically comprise macroelements, microelements, hormones, vitamins and sugars. It is selected to promote the growth and production of molecules of interest, namely according to the invention at least glycosylated flavonoids. The culture medium can also be selected, as seen above, to allow the growth of other secondary metabolites by the plant cells, in particular those naturally present in the cell line but in very small quantities, such as for example caffeic acids classics, in particular rosmarinic acid as shown in the examples given below. The extract obtained will advantageously include a whole set of secondary metabolites that can act synergistically and confer original and new properties in cosmetics.

Concerning the biomass treatment step, the recovery of the biomass manufactured in the bioreactor is carried out after a culture time of 7 to 21 days, preferably 10 to 14 days, so as to advantageously allow the highest amount of biomass to be produced and with high viability.

An additional drying step, in particular freeze-drying, of the cellular biomass that was recovered and freed from the supernatant, can be carried out, the advantage being that the biomass can thus be preserved in a more stable form in the long term. The dried biomass can be rehydrated or rediluted for its subsequent use, extemporaneously. A final step of extensive purification of the cellular extract to eliminate its debris is possible by all methods available industrially, by liquid-liquid partitioning or chromatography, in particular using an adsorbent resin, in order to concentrate the molecules of interest such as all the glycosylated flavonoid derivatives produced by calls. According to a second object, the present invention provides a plant extract obtainable by the method according to the invention and as described above.

According to other features, the extract according to the invention is characterized in that it is obtained from a plant of the Lamiaceae family and in that it comprises glycosylated flavonoids as secondary metabolites.

The extract obtained from Monarda didyma comprises in particular, as secondary metabolites, a set of glycosylated flavonoids derived from the added aglycone flavonoids. Preferably, the added aglycone flavonoids are part of the aglycone flavanone family, more preferably the naringenin.

The extract obtained from Lavandula angustifolia includes as secondary metabolites, a set of glycosylated flavonoids derived from the added aglycone flavonoids. Preferably, the added aglycone flavonoids are part of the aglycone flavonoid family, more preferably the naringenin.

The extract obtained from Abronia villosa includes as secondary metabolites, a set of glycosylated flavonoids derived from the added aglycone flavonoids. Preferably, the added aglycone flavonoids are part of the aglycone flavonoid family, more preferably the luteolin.

Thus, the undifferentiated or dedifferentiated plant cells obtained and/or the extract obtained can be used for the manufacture of an active ingredient for a cosmetic or dermatological composition intended to improve the general condition of the skin and its appendages.

According to a third object, the present invention provides a composition, in particular a cosmetic composition, comprising an extract according to the second object as active ingredient and a physiologically acceptable medium. The expression “physiologically acceptable medium” means according to the present invention, without imitation, an aqueous or hydroalcoholic solution, a water-in-oil emulsion, an oil in-water emulsion, a micro-emulsion, an aqueous gel, an anhydrous gel, a serum, a dispersion of vesicles or a powder.

“Physiologically acceptable” means that the compositions are suitable for topical use, in contact with mucous membranes, nails, scalp, hairs and skin of mammals, particularly human, without risk of toxicity, incompatibility, instability, allergic response, and others.

This “physiologically acceptable medium” forms what is commonly called the excipient of the composition. According to the invention, the physiologically acceptable medium can be an aqueous, hydroglycolic or hydroalcoholic medium, or formed by a water-in oil emulsion, an oil in-water emulsion, or a microemulsion. More preferably, it is hydroglycolic. More preferably, the physiologically acceptable medium is a mixture of water and glycerin.

Thus, according to the present invention it is possible to beautify or improve the appearance and general condition of the skin and/or appendages and to treat imperfections, thanks to the topical application to the skin of an effective quantity of at least one extract according to the invention and/or a composition comprising t, in a physiologically acceptable excipient, in a subject in need thereof.

According to a fourth object, the present invention provides the use of the extract according to the second object and/or of a composition according to the third object for a non-therapeutic cosmetic treatment of the skin and its appendages. Preferably according to the invention, the treatment is topical.

Several phenomena influence the skin quality and several parameters make it appear less homogeneous, for example wrinkles and fine lines, dryness, kiss of elasticity and pigmentation heterogeneities. These skin imperfections con disappear of be masked by aesthetic treatments beautifying the skin by making it mora homogeneous.

Results of in vitro and in vivo tests are given later in the description demonstrating beneficial cosmetic activities for the skin and its appendages for two examples of extracts produced according to the method of the invention from lines of Monarda didyma and Lavandula angustifolia.

These cosmetic effects can be envisaged according to the invention separately or in a combined manner, offering advantageously for example combined beautifying and sensory effects.

According to the invention, by “topical treatment” or “topical use” is meant an application which is intended to act at the place where it is applied: skin, mucous membrane and/or appendages.

The present invention preferably provides a use of an extract according to the invention derived from plant calls of the genus Monarda, and more particularly Monarda didyma, for at least one treatment chosen from:
an anti-aging treatment; and/or
an arti-seborrheic treatment; and/or
a moisturizing treatment; and/or
a treatment for strengthening the skin barrier and/or
a slimming treatment and/or
a soothing treatment.

More particularly, the anti-aging treatment proposed is adapted to act on pigmentation defects of the skin and more particularly according to the invention, the anti-aging treatment is advantageously adapted to treat white and/or brown age spots of the skin.

As the skin ages, two opposing phenomena cause brown spots and white spots that are often considered unsightly, Brown spots are due to hyperpigmentation while white spots are due to hypopigmentation.

By “hyperpigmentation” according to the present invention, is meant a spot rich in pigment(s) which is the consequence of the melanin production in excessive quantity in certain areas of the skin.

Conversely, by “hypopigmentation” is meant according to the present invention, a spot devoid of pigment. These spots result in an unsightly, non-homogeneous appearance of the skin colour.

Skin pigmentation originates from a very specific cell called melanocyte. This cell is responsible for the production of skin pigment, the melanin.

With aging and/or overexposure to the sun, melanocytes also age and are no longer as efficient. They are fewer in number, of lower quality and are distributed inhomogeneously. It is this inhomogeneous distribution which is responsible for brown and white spots, a sign of an aging skin.

In vitro test results are given below in the description on this particular activity improving the homogeneity of skin colour, more particularly:
preventing and/or treating loss of pigmentation of the skin, in particular with a view of preventing and/or treating areas where there are cutaneous white spots; and/or
preventing and/or treating hyperpigmentation of the skin, particularly for preventing and/or treating areas where there are cutaneous brown spots.

In vitro tests show that the Monarda didyma extract according to the invention is suitable for acting on several axes:
preserve the homeostasis and youthfulness of melanocytes by inhibiting the oxidative and free radical effects known to accelerate cell aging;
maintain the environment close to melanocytes in good conditions by reducing the production of proteases responsible for the destruction of matrix proteins such as collagen and elastin, and by increasing the essential elements synthesis for maintaining the homeostasis of keratinocytes and melanocytes such as collagens-I, -IV, -VII and -XVII but also elastin, laminins and hyaluronic acid. Indeed, the melanocytes physiology is closely linked to signals coming from neighbouring epidermal calls such as keratinocytes and fibroblasts. Likewise, the peripheral extracellular matrix of these melanocytes forms a microenvironment which dearly influences their production;
maintain the proper functioning of melanocytes and inhibit their entry into senescence by increasing the length of the dendrites of the melanocytes which distribute melanin to the keratinocytes and the quantity of spindle shaped melanocytes which is a criterion for the youth of the calls observed, and by inhibiting senescence melanocytes by reducing the presence of the enzyme SA beta-GAL (“senescence-associated beta-galactosidase”) strongly present in senescent calls, the DDK1 quantity identified as pro-senescence of the melanocyte, and the IL-6 and IL-8 production which are among the molecules whose secretion is increased by senescent cells compared to young cells.

An in vivo study presented below also confirms this particularly interesting and original activity on all senescence spots.

Preferably, the present invention also provides the use of an extract according to the invention derived from plant calls of the genus Lavandula or a composition containing it according to the invention, for at least one treatment chosen from:
an anti-aging treatment; and/or
a moisturizing treatment; and/or
a soothing treatment.

In vitro tests results are given below in the description demonstrating cosmetic activities beneficial to the skin and its appendages.

The extract according to the invention may be associated with one or more other active ingredients at effective concentrations that can act synergistically or additionally for reinforcing and achieving the desired effects described for the invention, such as the following agents: filtering radiations, in particular UVA, UVB, IR or issues from blue light, hydrating, moisturizing, humectant, calming, muscle relaxant, slimming, restructuring, firming, re-plumping, lifting, smoothing, acting on blood microcirculation, inflammation, free radicals, anti-aging, anti-line lines and wrinkles, lightening, acting on complexion, anti-glycation, anti-carbonylation, pro-pigmenting acting on stratum corneum, on dermal-epidermal junction, on HSP protein production, on firmness, elasticity and tone of skin, on hair growth or anti-regrowth (including eyelashes and eyebrows), on eye contours (dark circles and under eye bags), peptides, vitamins, etc.

More particularly, to improve or beautify the general condition of the skin, the extract according to the invention may be combined, without this list being exhaustive, with one or more active ingredients:
Moisturising and nourishing for example Vegesome Moist 24™ (Sederma): active ingredient containing a powder composed of below particles of Lycopodium clavatum loaded with an extract of Imperata cylindrica, which progressively hydrates the epidermis, and/or
Pigmenting, for example Silverfree™ (Sederma): active ingredient containing the Pal-PP peptide which stimulates several stops of the pigmentation method, and/or the Pal-PA peptide and/or generally the peptides with pro-pigmenting activity described in the applicant's patent application WO2014/090376, and/or TYR-OL™ and TYR-EXCEL™ proposed by Sederma and described in the patents FR2702766 et WO03/017066, based on oleyl tyrosine, and/or one or more of the noreugenin glycoside derivatives described by UNIVERSITY HAMBURG in the patent application WO2017/121445, and/or dihydroxyacetone, and/or one or more derivatives of the chromen-4-one family described by MERCK in the patent application WO2007/087966; and/or the pyrazolin-4,5-dione based compounds described by L′OREAL in the patent application WO97/35942; and/or
Preventing the signs of photo-aging, for example Venuceane™ (Sederma): active ingredient containing a biotechnological extract of Thermus thermophilus, which prevents visible signs of photoaging (spots, wrinkles, dryness . . . ), protects cell structures from UV damage and strengthens skin integrity; and/or
Skin tensor, for example Ferninage™ (Sederma): active ingredient containing an Engethandia chrysolepsis extract, which provides clastic and finning properties to the skin; and/or
Anti-pollution, for example Citystem™ (Sederma): active ingredient containing a biotechnological extract of Marrubium vulgare, which makes the skin soft and smooth, refines the skin texture, reduces the visibility of blackheads while leaving the skin radiant and purified.

Detailed examples are given in the galenic section below.

A composition according to the invention can be applied to the face, body, neckline, scalp, hair, eyelashes, body hair, in any form or vehicle known to those skilled in the art, in particular in the form of a solution, dispersion, emulsion, paste or powder, individually or as a premix or vehicled individually or as a premix in a bound form, incorporated or adsorbed in vectors such as macro-, micro-, or nanocapsules, macro-, micro- or, nanospheres, liposomes, oleosomes or chylomicrons, macro-, micro-, or nanoparticles or macro-, micro or nanosponges, micro- or nanoemulsions, or adsorbed on organic polymer powders, tales, bentonites, spores or exines, and other inorganic or organic supports.

In cosmetics, applications can be proposed in particular in the ranges of skin care for the face, body, hair and body hair and ranges of make-up treatments, in particular eyelashes, eyebrows and skin makeup ranges.

The composition may be incorporated onto a non-woven or woven material, with natural or synthetic fibres, wool, or any material intended to come into contact with skin and that can be used in clothing, including tights and socks; shorty, day or night underwear, tissues, handkerchiefs or fabric to exert its cosmetic effect via the contact skin/textile and enable continuous topical delivery (cosmetic textiles).

According to the invention, it is thus also providing a woven or non-woven fabric comprising the extract according to the invention for use in a non therapeutical cosmetic treatment.

The cosmetic formulations can enter in different product ranges for personal care and/or beauty products including skin care, cleaning, makeup, cleansing, sunscreen, artificial tanning, pre-shave, shaving or aftershave, moisturizer, humectant, emollient, conditioning exfoliating, astringent, depilatories or antiperspirant, deodorant, etc.

The Personal Care Products Council (“International cosmetic ingredient dictionary & handbook” published by the “Cosmetic, Toiletry, and Fragrance Association, Inc.”, Washington, D.C.) describes a nonlimited wide variety of cosmetic and pharmaceutical ingredients conventionally used in the skin care industry that can be used as additional ingredients in the compositions for the present invention, as long as they are physically and chemically compatible with the other ingredients of the composition and especially with the active ingredients of the present invention. Also, the nature of these additional ingredients should not unacceptably alter the benefits of the active ingredient of the invention. These additional ingredients can be synthetic or natural such as plants extracts or issued from a bio-fermentation process.

Further skin care actives that are particularly useful combined with the composition can be found in Sederma's commercial literature and on the website www.sederma.fr, in Croda's commercial literature and on the website www.croda.fr.

Commercially available actives widely used in cosmetic compositions can also be mentioned as examples: betain, glycerol, Actimoist Bio 2™ (Active organics), AquaCacteen™ (Mibelle AGCosmetics), Aquaphyline™ (Silab), AquaregulK™ (Solabia), Caroline™ (Greentech), Codiavelano™ (Biotech Marine), Dermaflux™ (Arch Chemicals, Inc), Hydra'Flow™ (Sochibo), Hydromoist L™ (Symrise), RenovHyal™ (Soliance), Seamoss™ (Biotech Marine), Essenskin™ (Sederma), Moist 24™ (Sederma), Argireline™ (commercial name of the acetyl hexapeptide-3 from Lipotec), spilanthol or an Acmella oleracea extract known under the trade name Gatuline Expression™, a Boswellia serrata extract known under the trade name Boswellin™, Deepaline PVB™ (Seppic), Syn-AKE™ (Pentapharm), Ameliox™ Bioxilift™ (Silab), PhytoCellTec™ Argan (Mibelle), Papilactyl D™ (Silab), Preventhelia™ (Lipotec), or one or more of the following active ingredients sold by Sederma: Subliskin™, Venuceone™, Moist 24™ Vegesome Moist 24™, Essenskin™, Juvinity™, Revidrat™, Resistem™, Chronodyn™, Kombuchka™, Chromocare™, Calmosensing™, Glycokin factor S™, Biobustyt™, Idealift™, Ceramide 2™, Ceramide A2™, Ceramide HO3™, Legance™, Intensin™, Prodizia™, Beautifeye™, Pacifeel™, Zingerslim™, Meiritage™, Sebuless™, Apiscalp™, Rutistem™, CitystemIN, Neonyca™, NG Insaporifiables de Beurre de Karto™ Majestem™, Hydronesis™, Poretect™, Amberstam™, Synchrolife™, Syfvorfree™, Feminage™, Ameyezing™, Revitalider™, or mixture thereof.

Among plant extracts (in the form of classical plant extracts or prepared by an in vitro process) can be used as additional actives, there may more particularly be mentioned extracts of Ivy, for example English Ivy (Hedera helix), of Bupleurum chinensis of Bupleurum falcatum, of arnica (Arnica montana L.), of rosemary (Rosmarinus officinalis N.), of marigold (Calendula officinalis), of sage (Salvia officinalis L.), of ginseng (Panax ginseng), of gingko biloba, of St.-John's-Wort (Hyperycum perforatum), of butcher's-broom (Ruscus aculeatus L.), of European meadowswest (Filipendula ulmaria L), of big-flowered Jarva tea (Orthosiphon stamincus Berth.), of artichoke (Cynara scolymus), of algae (Fucus vesiculosus), of birch (Betula alba), of green tea, of cola nuts (Cola nipida), of horse chestnut, of bamboo, of Centella asiatica, of heather, of fucus, of willow, of mouse-ear, of escine, of cangzhu, of Chyrsanthellum indicum, of the plants of the Armeriacea gens, Atractylodis platicodon, Sinomenium, pharbitidis, Flemingia, de Coleus as C. forskohlii, C. bluernei, C. esquirolii, C. scutelloroides, C. xanthartus and C. barbatus, such as the extract of root of Coleus barbatus, extracts of Balote, of Guioa, of Davallia, of Terminalia, of Barringtonia, of Trerna, of Antirobia, Cecropia, Argania, Dioscoreae such as Dioscorea opposita or Mexican, extracts of Ammi visnaga, of Siegesbeckia, in particular Siegesbeckia orientalis, vegetable extracts of the family of Ericaceae, in particular bilberry extracts (Vaccinium angustifolium) or Arctostaphylos uva ursi, Abe vara, plant containing sterols (e.g., phytosterol), Manjistha (extracted from plants of the genus Rubia, particularly Rubia cordifolia), and Guggal (extracted from plants of the genus Commiphora, particularly Commiphora mukul), kola extract, chamomile, red dover extract, Piper methysticum extract (Kava Kava™ from Sederma), Bacopa monieri extract (Bacocalmine™ from Sederma) and sea whip extract, extracts of Glycyrrhiza glabra, of mulberry, of melaleuca (tea tree), of Larrea divaricata, of Rabdosia rubescens, of Eugena gracillis of Fibraurea recisa hirudinea, of Chaparral sorghum, of sun flower, d'Enantia chlorantha, of Mitracarpe of Spermacocea genus, of Buchu barosma, of Lawsonia inermis L., of Adiantium capillus-veneris L., of Chelidonium majus, of Luffa cylindrica, of “Japanese Mandari” (Citrus reticulata Blanco var. unshiu), of Camelia sinensis, of Imperata cylindrica, of Glaucium flavum, of Cupressus sempervirens, of Polygonatum multiflorum, of Loveyly hemsleya, of Sambucus nigro, of Phaseolus lunatus, of Centaurium, of Macrocystis pyrifera, of Turnera diffusa, of Anemaurhena asphodelcides, of Portulaca pilosa, of Humulus lupulus, of Coffea aratica, of Hex paraguariensis, or of Globularia cordifolia, of Oxydendron arboxetum, of Albizzia julibrissin, of Zingimber zerumbet smith, of Astragalus membranaceus, of Atractylodes macrocephalae, of Plantago lanceolata, of Leontopodumalpinum (or edelweiss), of Mirabilis jalapa, of Apium graveolens, of Marrubium vulgare, Buddeja davidi Franch, Synringa vulgaris, Engelhardia chrysolepsis or orchids.

The compositions of the present invention may include peptides, including, without limitation, di-, tri-, tetra-, penta- and hexapeptides and their derivatives. According to a particular embodiment, the concentration of the additional peptide(s), in the composition, ranges from 1×10⁻³% and 20%, preferably from 1×10⁻⁴% and 10% preferably between 1×10⁻⁵% and 5% by weight.

The term “peptide” refers here to peptides containing 20 amino acids or less, their derivatives, isomers and complexes with other species such as a metal ion e.g., copper, zinc, manganese, magnesium, and others). The term ‘peptides’ refers to both natural peptides and (bio)synthetic peptides. It also refers to compositions that contain peptides and which are found in nature, and/or are commercially available.

Suitable dipeptides for use herein include but are not limited to Carnosine (beta-AH), YR, VW, NF, DF, KT, KC, CK, KP, KK, TT, PA, PM or PP.

Suitable tripeptides for use herein include, but are not limited to RKR, HGG, GHK, GGH, GHG, GKH, KPK, KFK, Kavak, KbalaAK, Kabuk, Kacak, KPK, KMOK, KMO₂K (MO₂ being a di-oxygenated sulfoxide methionine), PPL, PPR, SPR, QPA, LPA or SPA

Mention may also be made of non-limiting examples of tripeptides comprising:
a lysine with its lateral chain grafted with a proline as K(P)HG or K(P)GH, K(P)
a lysine with its lateral chain grafted with a pyroglutamic acid as K(Pyr)HG or K(Pyr)GH, K(PT):
a lysine with the amine function of the lateral chain acetylated as K(Ac)HG of K(Ac)GH, K(Ac);
a lysine with its lateral chain grafted with a hydroxyproline K(Hyp)HG or K(Hyp)GH, K(Hyp) as disclosed in WO2016/007965.

Suitable non limitative examples of tetrapeptides are KTFK (SEQ ID NO: 1), GOPR (SEQ ID NO: 2), RSRK (SEQ ID NO: 3), KTAK (SEQ ID NO: 4), KAYK (SEQ ID NO: S). KEYK (SEQ ID NO: 6), or TKPR (SEQ ID NO: 7). A suitable non limitative example of pentapeptide is KTTKS (SEQ ID NO: 8) and KTSKS (SEQ ID NO: 9) and examples of hexapeptides are GKTIKS (SEQ ID NO: 10) and VGVAPG (SEQ ID NO: 11).

Other suitable peptides for use according to the present invention can be selected, this list being not limitative, from: lipophilic derivatives of peptides, preferably palmitoyl (Pal) derivatives or myristoyl (Myr), and metal complexes as aforementioned (e.g., copper complex of the tripeptide HGG or GHK).

Preferred dipeptides include for example N-Palmitoyl beta-Ala-His, N-Acetyl Tyr-Arg hexadecylester (Calmosensine™, Idealift™, Sederma), Pal-KT, Pal-RT, Pal-PP and Pal-PA.

Preferred tripeptide derivatives include for example the copper derivative of HGG (Lamin™ from Sigma), Pal-GKH and Pal-GHK (from Sederma), Lipospondin (N-Elaidoyl-KFK) and its analogs of conservative substitution, N-Acetyl-RKR-NH2 (Peptide CK+), N-Biot-GHK (from Sederma), Pal-KAvak, Pal-KbataAlak, Pal-KALuK, Pal-KAcak, or Pal-KMOCK (Matrixyl®synthe6® from Sederma), Pal-KVK (Syn-Coll™ of DSM), and derivatives thereof.

Mention may also be made here of the anti-aging tripeptides of general formula X-Pro*-Pro*-Xaa-Y described in WO2015181698 Xaa selected from Leu, Arg, Lys. Ala, Ser, and Asp, at the N-terminus, X chosen from H, —CO—R¹ and —SO₂—R¹ and at the C-terminal end Y chosen from OH, OR¹, NH₂, NHR¹ or NR²R², R¹ and R² being, independently of one another, chosen from a alkyl, aryl, aralkyl, alkylaryl, alkoxy and aryloxy group, which may be linear, branched, cyclic, polycyclic, unsaturated, hydroxylated, carbonylated, phosphorylated and/or sulfurized, said group possibly possessing in its backbone a heteroatom particularly O, S and/or or N, and Pro* corresponding to Proline, an analogue or derivative thereof; comprising, for example, Myr-PPL-OH and Myr-PPR-OH.

Here can further be cited also the propigmenting and/or pro-MEC peptides and tripeptides of general Formula X-(Xaa₁)n-Pro*-Xaa₂-Y disclosed in WO2014/090376, with n=0, 1 or 2, Kas, an hydrophobic aminoacid selected from Ala, Val, Met, Lou, Iso, Phe, Pro, and analogues and derivatives thereof; or a polar aminoacid selected from Ser, Thr, Tyr, Asp, Glu and analogues and derivatives thereof; and when n=2 the to aminoacids Xaa, being the same or different; Xaa, being an hydrophobic aminoacid selected from Ala, Val, Met, Lou, Iso, Phe, and analogues and derivatives thereof, or a basic aminoacid selected from Arg, Lys, His, and analogues and derivatives thereof; at the N terminal end X being selected from H, —CO—R₁ and —SO₂—R₁; at the C terminal end Y being selected from OH, OR₁, NH₂, NHR₁ or NR₁R₂; R₁ and R₂ being, independently from each other, selected from an alkyl aryl, aralkyl, alkylaryl alkoxy et aryoxy group, that can be linear, branched, cyclic polycyclic, saturated, unsaturated, hydroxylated, carbonylated, phosphorylated and/or sulfured, said group having or not an O, Sandier N heteroatom in its skeleton and Pro′ corresponding to a Proline, analogue or derivative thereof; comprising for example the following peptides Pal-SPR-OH, Pal-PPR-OH, Pal-QPA-OH, Pal-LPA-OH, My-SPA-OH, Pal-PM OH, Pal-PA-OH and Pal-PP-OH.

Suitable tetrapeptides derivatives for use as additional peptides according to the present invention include but are not limited to Ela-KTAK (SEQ ID NO: 12), Els-KAYK (SEQ ID NO: 13). Ela-KFYK (SEQ ID NO: 14), Pal-GOPR (SEQ ID NO: 15) or Pal-KTFK (SEQ ID NO: 16).

Suitable pentapeptides derivatives for use as additional peptides herein include, but are not limited to, Pal-KTTKS (SEQ ID NO: 17) (MATRIXYL™, Sederma), Pal-KTSKS (SEQ ID NO: 19), Pal-YGGFXaa (SEQ ID NO: 10) with Xaa being Trp, Phe, Tyr, Tic, 7 hydroxy-Tic of Tpi, or mixtures thereof.

Suitable hexapeptides derivatives for use herein include, but are not Invited to, Pal-HLDIIXas (SEQ ID NO: 20) with Xaa being Trp, Phe, Tyr, Tic, T-hydroxy-Tic or Tpi, Pal-GKTTKS (SEQ ID NO: 21), Pal-VGVAPG (SEQ ID NO: 22) (DERMAXYL™, Sederma), or mixtures thereof.

The preferred compositions available commercially and sold by Sederma:
tripeptides or a derivative include Biopeptide-CL™, Maxi Lip™, or Procapil™ containing GHK;
tetrapeptides or a derivative include RIGIN™, Esuliss™ containing Pal GOPR (SEQ ID NO: 15) and an excipient, Crystalde™ containing Pal-KTFK (SEQ ID NO: 16) vehicle (solvated in microemulsion);
pentapeptide or a derivative as Matrixyl™ containing Pal-KTTKS (SEQ ID NO: 17).

Can also be mentioned:
the mixture of Pal-GHK and Pal GOPR (SEQ ID NO: 15) (Matrixyl™ 3000), and
the mixture of Pal-GHK and Pal VGVAPG (SEQ ID NO: 22) (Biobustyl™)

The following marketed peptides can be mentioned as well as additional active ingredients:
Vialux™ (INCI name=Pentapeptide-3 (synthetic peptide comprising alarine, arginine, isoleucine, glycine and proline)), Syn-ake™ (bots-Ala-Pro-Dab-NH-Bzl) or Syn-Col™ (Pal-Lys Val-Lys-OH) marketed by Pentapharm;
Argireline™ (Ac-Glu-Glu-Met-Gin-Arg-Arg-NH: (INCI name=Acetyl hexapeptide-3) (SEQ ID NO: 23), Leuphasyl™ (Tyr-D-Ala-Gly-Phe-Leu) (SEQ ID NO: 24), Aldenine™ (Gly-His-Lys), Tryfagen™ (INCI name=Pseudoalteromonas Ferment Extract, Hydrolyzed Wheat Protein, Hydrolyzed Soy Protein, Tripeptide-10 Citrulline (reaction product of Citrulline and Tripeptide 10 (synthetic peptide constituted of aspartic acid, isoleucine and lysine)): Tripeptide-1), Eyesaryl™ (Ac-bets-Ala-His-Sar-His) (SEQ ID NO: 25), Serilesine™ (Ser-Ile Lys-Val-Ala-Val) (SEQ ID NO: 26) or Decorinyl™ (INCI name: Tripeptide-10 Citruline=reaction product of Citrusline and Tripeptide-10 (synthetic peptide constituted of aspartic acid, isoleucine and lysine) marketed by Lipotec
Collaxyl™ (Gly-Pro-Gin-Gly-Pro-Gin (SEQ ID NO: 27) or Quintascine™ (Cys-Gly) marketed by Vincience;
Cytokinol™ LS (casein hydrolysate) marketed by Les Laboratories Serobiologiques/Cognis;
Kollaren™ (Gly-His-Lys), IP2000™ (Pal-Val-Tyr-Val) or Melprene™ (INCI name=Monoluorcheptapeptide-1: reaction product of acetic acid and a synthetic peptide comprising arginine, glycine, glutamic acid, histidine, norleucine, p-fluorophenylalanine and tryptophan) marketed by l'institut Européen de Biologie Cellulaire;
Noutrazen™ (Pal-His-D-Pha-Any-NH₂) marketed by Innovations; or
BONT-L-Peptide™, Timp-Peptide™ or ECM Mouline™ marketed by Infinitec Activos.

It is also possible to envisage combining the invention with one or more cyclic peptides, in particular those extracted from linseed oil described in the Applicant's patent application WO2019/149450.

The extract according to the invention of composition containing it can be preferentially combined with at least one compound chosen among vitamin B3, compounds as niacinamide or tocopherd, relincids compounds as retinol, hexanidine, α-lipoic acid, resveratrol or DHEA, hyaluronic acid, ceramides, peptides, in particular N-acetyl-Tyr-Ang-O-hexadecyl, Pal-VGVAPG (SEQ ID NO: 22), Pal-KTTKS (SEQ ID NO: 17), Pal-KTSKS (SEQ IS NO: 18), Pal-GHK, Pal-KMO₂K. Pal-GQPR (SEQ ID NO: 15) and Pal-K(P)HG (MATRIXYLT™ Morphomics™, Sederma) that are classic active agents used in topical cosmetic or dermo-pharmaceutical compositions.

The extract or the composition according to the invention can be applied locally to the targeted areas.

The effective amount of the extract according to the invention, that is to say its dosage, depends on the destination of the composition. It depends on various factors, such as the age, the condition of the patient, the severity of the disorder or disease and the administration made. An effective amount means a non-toxic amount enough to achieve the desired effect.

In a cosmetic composition according to the invention containing at least the extract according to the invention, to be present in an effective amount, is generally present in an amount ranging from 0.000001% (0.01 ppm) and 15% (150 000 ppm) based on the total weight of the composition, preferably ranging from 0.00001% (0.1 ppm) and 10% (100 000 ppm), depending on the destination of the composition and the more or less pronounced desired effect.

More preferably, the effective amount is between 0.0001% (1 ppm) and 0.01% (100 ppm) based on the total weight of the composition, which is much lower than current market standards.

All percentages and ratios used herein are by weight of the total composition and al measurements are made at 25° C. unless it is otherwise specified.

For indication, for a cosmetic face treatment, the European standard dosage of a cream is 2.72 mg/cm²/day/person and for a cosmetic body treatment the European standard dosage of a lotion is 0.5 mg/cm²/day/person.

According to other features, the cosmetic treatment method according to the invention can be combined with one or more other treatment methods targeting the skin such as luminotherapy, heat or aromatherapy treatments.

It is possible to offer devices with several compartments or kits may be proposed to apply the method described above which may include for example and non-restrictively, a first compartment containing a composition comprising the extract according to the invention, and in a second compartment a composition containing another active ingredient and/or excipient, the compositions contained in the said first and second compartments in this case being considered to be a combination composition for simultaneous, separate or stepwise use in time, particularly in one of the treatment methods recited above.

### 1. Examples of Preparation of Extracts According to the Invention

### 1.1. From the Monarda didyma Plant

### 1.1.1. Creation of a Cell Line

Selected Monarda didyma leaves are collected, washed and cut into small pieces of a few millimetres, to produce numerous explants. After a series of decontamination treatments, the leaf samples are placed on a panel of agar culture media, in order to induce callogenesis (formation of callus).

After an appropriate period, an aggregate of dedifferentiated cells, called callus, forms, which is transferred to a larger volume of fresh culture medium so that it can multiply. A certain number of subcultures (transfers to a fresh culture medium) are carried out to stabilize the cel line, that is to say until it presents a satisfactory and constant proliferation rate, conservation of the phenotype, a constant content of bioactive compounds of interest (primary and secondary metabolites).

The cell line is then subjected to's selection step which consists of cultivating the calls for an appropriate period, taking the aggregates of cells formed and inoculating them in a liquid culture medium for a period allowing the multiplication of the cells in small dusters and/or individualized. The culture medium used is of the Murashige and Skoog type.

The best call line is the one which makes it possible to obtain as quickly as possible and reproducibly a satisfactory biomass having an optimal content of selected metabolites, the best biological activity and a homogeneous phenotype. Rosmarinic acid is one of the secondary metabolites of interest chosen.

### 1.1.2. Obtaining a Biomass of Dedifferentiated Monarda didyma Cells Including the Desired Secondary Metabolites

The call line prepared as described above or a pre-existing and conserved line is used as starting material. Firstly, the Monarda didyma line is multiplied to obtain a biomass having a sufficient quantity of dedifferentiated calls in order to carry out production step at a large-scale.

The following steps are implemented
a) Inoculation of the selected line in a liquid medium and culture for a sufficient time to obtain a quantity of biomass multiplied by 2 or even 3 times compared to the original biomass;
b) Optionally transfer of the suspension obtained in a) into a fresh liquid medium and cultured again for a sufficient time for a quantity of biomass multiplied by 2 or even 3 times compared to the original biomass;
c) Optionally, repetition of step b);

Steps a) to c) constitute the so-called pre-culture step. The culture medium used is of the Murashige and Skoog types.

The transfer of the cell suspensions obtained in stages a) to c) is carried out in a bioreactor with a specific production medium deficient in hormones, in which the biomass will proliferate in particular with an exponential phase. Once the desired biomass level is sufficient, in the middle of the exponential phase, between 20 and 90 mg/L of naringenin is added between 0.1 and 15 mg/L of methyl jasmonate as the elicitor. The culture medium used at this stage is type 85 from Gamborg. This type of medium makes it possible to greatly increase the quantity of rosmarinic acid produced by plant cells. The culture under these conditions is left for a sufficient time to obtain a cellular biomass containing the secondary metabolites of interest, that is to say the glycosylted derivatives of naringenin which are the subject of the invention and rosmarinic acid in sufficient quantities. This stop d)-called bioreactor culture step.

### The Bioreactor:

Volume: 5 to 100 times larger than the volume of biomass used as inoculum; internal surface of the bioreactor smooth and uniform.

### Growing Conditions:

Culture medium: medium comprising mineral salts (solution of macroelements and microelements), vitamins, plant hormones as well as sucrose. Plant agar is added to the solid media for the line creation step.

Temperature: between 15° C. and 35° C., preferably between 20° C. and 30° C. and even more preferably at 25° C. Duration: between 7 and 21 days with stirring of the biomass so that it is optimally aerated, preferably between 10 and 14 days.

Agitation of the biomass: the biomass is optimally aerated, and at the same time, it is agitated either by internal means of by external means. Low and efficient agitation is maintained in the final step when the biomass is in large quantity. For the purposes of the present invention, suitable means of stirring internally are propellers rotating between 0.10 and 0.75 mis, preferably at 0.21 m/s, or externally means of stirring orbital agitation rotating preferably between: 40 and 200 rpm and preferably at approximately 110 rpm.

Oxygenation: Normally accomplished using sterile air or gas mixtures containing 10% to 100% by volume of oxygen.

### 1.1.3. Recovery of the Biomass of Dedifferentiated Cells

Biomass consists of cells in suspension, partially lysed or whole cells, in the form of clusters or individuals. Filtration is carried out to eliminate the remaining culture medium (supernatant) and recover the call biomass. Optionally, before the filtration step, on antioxidant is added to the biomass to protect the secondary metabolites from oxidation.

The recovered cellular biomass can be characterized by HPLC/UV. To this aim, the calls are extracted in an ethanol/water mixture (70/30 by volume) for analysis.

The HPLC/UV analysis of this extract shows that the cellular biomass comprises between 200 and 500 ppm of said glycosylated flavonoid derivatives between 1000 and 2000 ppm of rosmarinic acid by weight relative to the total weight of said extract.

### 1.1.4. Treatment of the Biomass of Dedifferentiated Cells to Obtain an Extract According to the Invention

The contents of the cells are extracted by osmotic diffusion by adding glycerin and separation of the liquid and solid phases by centrifugation or filtration or equivalent, in order to obtain a cellular extract free of cellular debris. Optionally, the extraction of cell contents can also be done by grinding, mechanical or chemical lysis of the cells. The Monarda didyma extract according to the invention then comprises the intracellular content of dedifferentiated plant calls, freed of its cellular debris.

The active compounds contained in the extract obtained are characterized analytically by chromatography. The analytical results are given below in the description at point 5.

The following steps are optional:
1. Drying of the extract in particular by lyophilzation, zeodratation or atomization to allow greater stability of the interest compounds, improve long term storage without having to add preservatives.
2. High pressure homogenization of cellular biomass: allows a reduction in the size of cellular aggregates.
3. Purification of the call extract to increase the content of glycosylated flavonoids and rosmarinic acid, for example by an additional ethanol water extraction (70/30 by volume).

### 1.2. From the Lavandula angustifolia Plant or the Abronia villosa Plant

The same method steps as described for Monarda didyma above are implemented, except that the aglycone flavonoid precursor used here is luteolin. The luteolin quantity added is between 200 mg′L and 1000 mg/L.

In the same way as for Monarda didyma, at the end of the process an extract of plant origin is obtained characterized by the presence of glycosylated derivatives of luteolin in the intracellular content of the harvested plant cells.

The active compounds contained in the extract obtained are characterized analytically by chromatography. The analytical results are given below in the description at point 5.

### 2. Example of Preparation of an Active Ingredient for Cosmetic Use

Depending on its concentration of secondary metabolites, the extract according to the invention, as prepared according to the examples above, con constitute as such an active ingredient, the glycerol having been used for osmotic diffusion advantageously constituting the physiologically acceptable medium.

To form the active ingredient, the extract can also be diluted to reduce its concentration of secondary metabolites in a physiologically acceptable medium. This can be identical to the medium used for osmotic diffusion, that is to say here glycerol, or not identical, for example butylene glycol alone or mixed with glycerol.

The ingredient thus formed can comprise for example 20% by weight of fresh biomass extract of dedifferentiated calls, in glycerol (approximately 20 to 80%), said ingredient containing between 0.002% and 4% of said glycosylated flavonoid derivatives and 0.002% and 4% rosmarinic acid by weight relative to said ingredient.

This ingredient can then be used to prepare cosmetic formulations such as those for example sat out below. An effective quantity of this ingredient represents between 03% and 15%, preferably between 196 and 5%, more preferably between 2% and 4% and generally 3% by weight of said formulation.

### 3. Examples of Cosmetic Formulations

An example of a cosmetic formulation is given below containing the extract according to the invention as described above. Furthermore, these formulations can also contain additional cosmetic active ingredients, the latter coming for each case in support and/or complement of the activity of the active ingredient according to the invention. These ingredients can be of any class according to their's) function(s), site of application body, face, neck, chest, hands, etc.), the desired and effect and the target consumer, for example anti-wrinkle, moisturizing, anti-dark circles, firming, anti-glycation, volumizing, soothing, muscle relaxant, anti-redness, detoxifying, etc.

### Cream Form

TABLE 1

| Raw materials | % | INCI name |
|---|---|---|
| Phase A | | |
| H₂O | qsp | Water |
| Carbomer | 0.40 | Carbomer |
| Phase B | | |
| Crodamol ™ OSU | 7.00 | Diethythexyl Succinate |
| Crodafos ™ CES | 4.00 | Cetearyl Alcohol (and) Dicetyl |
| | | Phosphate (and) Ceteth-10 Phosphate |
| Crodacol ™ CS90 | 1.50 | Cetearyl Alcohol |
| Crodamol ™ AB | 1.50 | C12-15 Alkyl Benzoate |
| Brij ™ S10 | 1.20 | Steareth-10 |
| Brij ™ S2 | 0.40 | Steareth-2 |
| Phase C | | |
| Glycerin | 2.50 | Glycerin |
| Caprylyl glycol | 0.50 | Caprylyl glycol |
| Phase D | | |
| Phenoxyethanol | qs | Phenoxyethanol |
| Phase E | | |
| Potassium sorbate | qs | Potassium sorbate |
| Phase F | | |
| H₂O | 4.50 | Water |
| Sodium hydroxide to 30% | 0.45 | Sodium hydroxide |
| Phase G | | |
| Ingredient according to the | 3.00 | / |
| invention | | |

### Protocol:

Swell the carbomer in water (phase A) then heat Weigh and hast B. Mell C and leave to cod. Add D to C, then C+D to A mix. In C+D+A, add B, then E, then F, then G, homogenize.

### Serum Form

TABLE 2

| Raw materials | % | INCI names |
|---|---|---|
| Phase A | | |
| H₂O | qsp | Water |
| Pentylene glycol | 5.00 | Pentylene glycol |
| Propanediol | 3.00 | Propanediol |
| Amigum | 0.40 | Sclerotium Gum |
| Potassium Sorbate | qs | Potassium Sorbate |
| Phase B | | |
| Glycerin | 7.00 | Glycerin |
| Xanthan gum | 0.60 | Xanthan gum |
| Phase C | | |
| NatraGem ™ S140 NP | 1.00 | |
| Crodamol ™ SSA | 3.00 | Polyglyceryl-4 Laurate/Sebacate (and) |
| | | Polyglyceryl-6 Caprylate/Caprate |
| | | (and) Aqua |
| | | Decyl Isostearate (and) |
| | | Isostearyl Isostearate |
| Phase D | | |
| H₂O | 0.25 | Water |
| Lactic acid | 0.025 | Lactic acid |
| Phase E | | |
| Ingredient according | 3.00 | / |
| to the invention | | |

### Protocol:

Weigh and mix A. Weigh and mix B. Add B to A while stirring, mix. Weigh and mix C. Add C to B+A while stirring, mix. Weigh and mix D. Add D to B+A+C, then E, then F, homogenize.

Melt C and leave to cool. Add D to C, then C+D to A, mix. In C+D+A, add B, then E, homogenize.

Examples of Ingredients that can be Added to these Formulations (Marketed by SEDERMA):

VENUCEANE™: active ingredient containing a biotechnological extract of Thermus thermophilus, which prevents visible signs of photoaging (spots, wrinkles, dryness, etc.), protects cellular structures from damage caused by UV radiations and strengthens the integrity of the skin; AQUALANCE™; osmoprotective moisturizing active ingredient CALMOSENSINE™; calming active ingredient CRYSTALIDE™: active ingredient more specifically for soothing the epidermis.

### 4. Analytical Tests

The extract used for the analytical tests is that described in point 2.1.3. above. The extraction of the calls is carried out by mixing in an ethanol/water solution (70/30 by volume), which is then filtered and then diluted before being injected into HPLC/UV.

The assay is carried out on a Waters HSS C18 column, with an elution gradient of 15 minutes, composed with an aqueous ammonium formate phase SOM native pH, water and acetonitrile.

It is indeed the intracellular content, that is to say what was produced by the plant cells, which is characterized.

### 4.1. Comparison During the Method, Before Adding the Precursor and after Metabolizing it

The objective is to show that a biotransformation has occurred by plant cells. To this aim, for the two examples of plants given above, Monarda didyma and Lavandula angustifolia, a chromatogram is carried out on an extract made from a sample of biomass taken in the bioreactor just before or just after the addition of the precursor, and a chromatogram is carried out on an extract made from a sample of the biomass at 7 days. For each plant, the two chromatograms are compared.

### Results

### 4.1.1. Monarda didyma

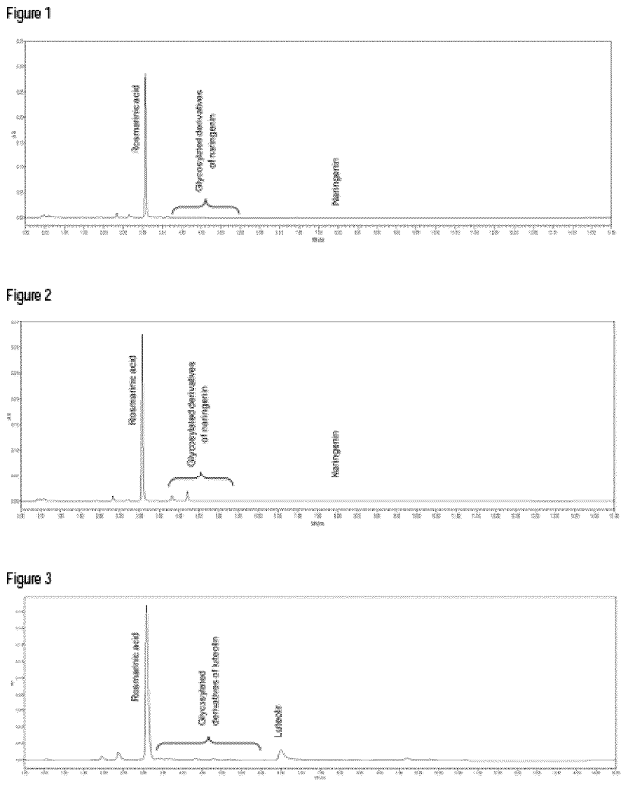

FIG. 1: represents a chromatogram monitoring the secondary metabolites produced by a cellular biomass of Monarda didyma just before addition of naringenin.

FIG. 2: represents a chromatogram of biomass monitoring as in FIG. 1 but seven days after the addition of naringenin, this figure illustrating the bioconversion of naringenin into a set of glycosylated derivatives thereof.

On the chromatogram in FIG. 1, the peak of rosmarinic acid present at 3.2 minutes is observed. This is consistent with expectations from a cell culture of Monarda didyma, rosmarinic acid being known as a secondary metabolite in the plant.

On the chromatogram in FIG. 2, the peak corresponding to rosmarinic acid (3.2 minutes) is still observed but also several peaks between 3.5 and 5.5 minutes which were not present in the chromatogram of FIG. 1 appeared. It was possible to determine that they corresponded to a set of glycosylated derivatives of naringenin. Furthermore, the retention time of naringenin is 8 minutes. It can be seen that this peak does not appear in FIG. 1 or FIG. 2.

This clearly shows that the dedifferentiated plant cells of Monarda didyma integrated and then metabolized the naringenin added to the culture medium into a set of glycosylated derivatives of naringenin.

### 4.1.2. Lavandula angustifolia

FIG. 3: represents a monitoring chromatogram as in FIG. 1 for a cellular biomass of Lavandula angustifolia a few minutes after addition of luteolin.

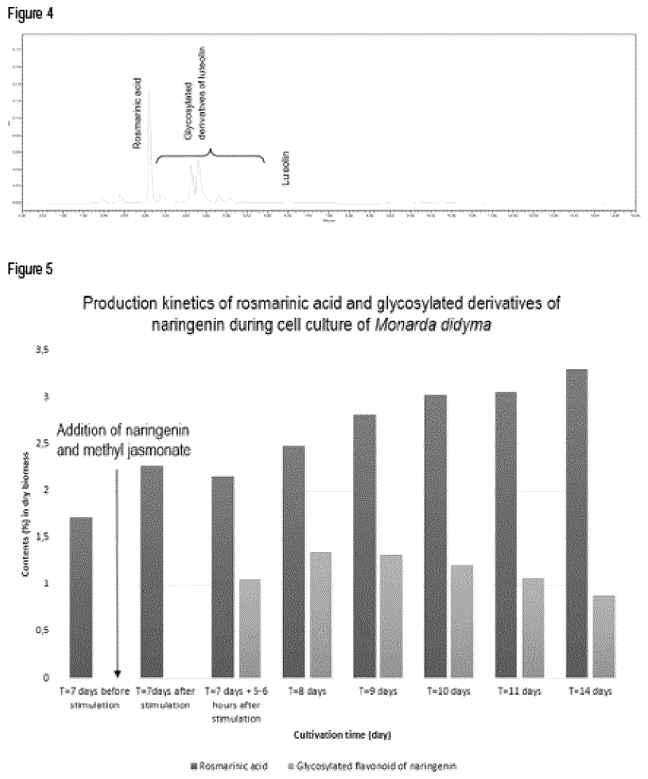

FIG. 4: represents a biomass monitoring chromatogram as in FIG. 3 but eight days after the addition of luteolin, this figure illustrating the bioconversion of luteolin into a set of glycosylated derivatives thereof.

On the chromatogram in FIG. 4, a peak at 3.10 minutes corresponding to rosmarinic acid is observed and several peaks between 3.30 and 6.00 minutes corresponding to a pool of glycosylated derivatives of luteolin and a final peak at 6.50 minutes corresponding to luteolin are also observed.

On the chromatogram in FIG. 3, only the peaks of rosmarinic acid and luteolin are present. The peak corresponding to luteolin is 0.0125 arbitrary units in FIG. 3 and 0.005 arbitrary units in FIG. 4.

The dedifferentiated plant cells of the Lavandula angustifolia extract according to the invention integrated and metabolized a large part of the luteolin added to the biomass into a set of glycosylated derivatives of luteolin.

It can also be soon in this case that the cells of Lavandula angustifolia were capable of metabolizing a very large luteolin quantity, which advantageously makes it possible to obtain an extract very concentrated in secondary metabolites.

### 4.2. Effect of the Addition of the Aglycone Flavonoid and/or an Elicitor on the Obtained Extract after Metabolization

### 4.2.1. Monarda didyma

The tests were carried out on an extract of Monarda didyma obtained by in vitro cel culture under different conditions. Only one type of growing medium is used here, such as Murashige and Skoog.

The measurements were made on the product resulting from the method. The objective is to compare the contents of glycosylated derivatives of naringenin and rosmarinic acid obtained.
Ext 1: An extract according to the invention obtained with the addition of naringenin;
Ext 2: An extract according to the invention obtained with the addition of naringenin and methyl jasmonate (elicitor);
Ext 3: An extract with added methyl jasmonate, but no aglycone flavonoid;
Ext 4: An extract with the addition of neither aglycone flavonoid nor elicitor.

### Results

Content of Rosmarinic Acid and Glycosylated Naringenin Derivatives in the Different Extracts of Monarda didyma:

TABLE 3

| | | Glycosylated |
|---|---|---|
| | Rosmariric | derivatives |
| | acid | of naringenin |
| Ext 1 according to the | 20 ppm | 54 ppm |
| invention (+ naringenin) | | |
| Ext 2 according to the invention | 280 ppm | 334 ppm |
| (+ naringenin and+ methyl jasmonate) | | |
| Ext 3 (+methyl jasmonate) | 302 ppm | <LOD |
| Ext 4 (neither naringenin nor | 91 ppm | <LOD |
| methyl jasmonate) | | |

LOD: limit of detection

The results show that the extracts contained without the addition of naringenin (Ext 3 and 4) do not present glycosylated flavonoids compare to the extracts according to the invention obtained with the addition of naringenin (Ext 1 and 2). The effect of elicitation by methyl jasmonate (Ext 3) is also visible with a greatly increased level of rosmarinic acid (91 ppm to 302 ppm), as well as this effect on the extract obtained with addition of naringenin (Ext 2) compared to the extracts obtained without addition (Ext 1 and 4).

A strong increase in the level of rosmarinic acid (20 to 200 ppm) and a very strong increase in glycosylated flavonoids (54 to 334 ppm) in the extract according to the invention obtained with the addition of naringenin and methyl jasmonate (Ext 2) compared to the extract according to the invention obtained with addition only of naringenin (Ext 1) are also noticed. This shows the advantage of also adding elicitation to the method according to the invention, not only on the level of rosmarinic acid but also on the level of glycosylated flavonoids.

### 4.2.2. Lavandula angustifolia

Tests were also carried out on an extract of Lavandula angustifolia obtained by in vitro culture under different conditions. Only one type of growing medium is used here, such as Murashige and Skoog. The measurements were made on the product resulting from the method. The objective is to compare the contents of glycosylated derivatives of luteolin and rosmarinic acid obtained.
Ext 1: An extract according to the invention obtained with the addition of luteolin;
Ext 2: An extract according to the invention obtained with the addition of luteolin and methyl jasmonate (elicitor):
Ext 3: An extract with added methyl jasmonate, but no aglycone flavonoid;
Ext 4: An extract with the addition of neither aglycone flavonoid nor elicitor.

### Results

### Content of Rosmarinic Acid and Glycosylated Derivatives of Luteolin in the Different Extracts of Lavandula Angustifolia:

TABLE 4

| | | Glycosylated |
|---|---|---|
| | Rosmarinic | derivatives |
| | acid | of luteolin |
| Ext 1 according to the invention (+ luteolin) | 1293 ppm | 1572 ppm |
| Ext 2 according to the invention | 3933 ppm | 2109 ppm |
| (+ luteolin and+ methyl jasmonate) | | |
| Ext 3 (+methyl jasmonate) | 3594 ppm | <LOD |
| Ext 4 (neither luteolin nor methyl jasmonate) | 1450 ppm | <LOD |

The results show that the extracts obtained without the addition of luteolin (Ext 3 and 4) do not present glycosylated flavonoids compare to the extracts according to the invention obtained with the addition of luteolin (Ext 1 and 2). The effect of elicitation by methyl jasmonate (Ext 3) can also be seen with a greatly increased level of rosmarinic acid (1450 ppm to 3534 ppm), as well as this effect on the extract obtained with addition of luteolin (Ext 2) compared to the extracts obtained without addition (Ext 1 and 4).

A strong increase in the level of rosmarinic acid (1293 to 3933 ppm) and a very strong increase in glycosylated flavonoids (1572 to 2100 ppm) in the extract according to the invention obtained with the addition of luteolin and methyl jasmonate (Ext 2) compared to the extract according to the invention obtained with addition only of luteolin (Ext 1) are also observed. This shows the advantage of also adding elicitation to the method according to the invention, not only on the level of rosmarinic acid but also on the level of glycosylated flavonoids.

### 4.2.3. Abronia villosa

Tests were also carried out on extract of Abronia villosa obtained by in vitro cell culture under different conditions. Only one type of growing medium is used here, such as Murashige and Skoog. The measurements were made on the product resulting from the method. The objective is to compare the content of glycosylated derivatives of luteolin obtained.
Ext 1: An extract according to the invention obtained with the addition of luteolin;
Ext 2: An extract with the addition of neither aglycone flavonoid nor elicitor.

### Results

Content of Glycosylated Derivatives of Luteolin in the Different Abronia villosa Extracts:

TABLE 5

| | Glycosylated derivatives |
|---|---|
| | of luteolin |
| Ext 1 according to the invention (+ luteolin) | 1764 ppm |
| Ext 2 (neither luteolin nor methyl jasmonate) | <LOD |

The results show a very strong increase in glycosylated flavonoids in the extract according to the invention obtained with the addition of luteolin (Ext 1) compared to the extract obtained without addition of luteolin (Ext 2) which does not contain glycosylated flavonoids.

### 4.2.4. Conclusion

The addition of aglycone flavonoid to the biomass therefore makes it possible to produce glycosylated flavonoids do not present in the plant cels of the original biomass. The addition of methyl jasmonate makes it possible to increase the quantity of rosmarinic acid and the quantity of glycosylated derivatives of the precursor flavonoid.

### 4.3. Kinetics of Production of Molecules of Interest During the Same Cell Culture

The objective is to compare the quantities of aglycone flavonoids and glycosylated derivatives thereof over time during the preparation of an extract of Monarda didyma obtained by in vitro cel culture according to the invention, before and after addition of the aglycone flavonoid.

### Results

The bar graph of the FIG. 5 illustrates the production kinetics of rosmarinic acid and glycosylated derivatives of naringenin during call culture, with the contents in the dry biomass on the ordinate and different culture times in days on the abscissa. All percentages are given by weight relative to the total weight of the dry extract.

The graph shows at T=7 days of culture after addition of methyl jasmonate and naringenin the extract obtained contains only rosmarinic acid which was increased from 1.7% before addition to 2.3% after addition.

A few hours after the addition of naringenin (T=7 days of culture+5-6 hours after addition), glycosylated derivatives of naringenin appear (around 1.19%). Then this quantity increases (1.35% at T=8 days of culture), reaches a plateau (1.32% % at T=0 days of culture) before decreasing (1.2% at T=10 days of culture and 0.9% at T=14 days of culture). For rosmarinic acid, it increases over time after the addition of methyl jasmonate (from 1.796 at T=7 days of culture before addition to 3.3% at T=14 days of culture).

This kinetic monitoring also makes it possible to show that the addition of aglycone flavonoid to the culture medium causes the plant calls to produce glycosylated derivatives of these which were not present in the initial biomass. It can be seen here that the addition of an elicitor makes it possible to increase the quantity of rosmarinic acid, a molecule already present in the initial biomass, that is to say before addition of aglycone flavonoid and elicitor.

### 4.4. Mechanism Explanation of the Production of Molecules of Interest by Monarda didyma Extract

### Principle

The aim of this study is to explain the production mechanism of secondary metabolites, in particular the production of glycosylated derivatives, by Monarda didyma cels.

A number of hypothesises were put forward:
1) The aglycone flavonoid can reveal particular metabolic pathways which were indeed present, but which are not yet used due to lack of precursors. If the enzyme responsible to convert the aglycone flavonoid is naturally present in the call, it will be found outside the cel when it is ground. So, the enzyme, whether inside or outside the cell, can convert the aglycone flavonoid into a glycosylated derivative. Therefore, this case can be observed on whole calls (fest condition) and crushed cells (third condition) that have converted the naringenin into a glycosylated derivative of the aglycone flavonoid. In the supernatant (second condition), no glycosylated derivatives should be found since the enzyme can be present only in the cell and not directly in the supernatant.
2) Aglycone flavonoid can play the role of epigenetic probe and as a precursor. The epigenetic probe is capable of sending signals to the DNA of cells, thus making it possible to generate and/or mobilize certain particular enzymes capable of metabolising the probe. The precursor is the molecule used for the conversion. Contrary to the first hypothesis, the enzyme cannot be generated and/or mobilised outside of the cell, since DNA is not alive outside the cell. So, the aglycone flavonoid cannot convert into a glycosylated derivative in the crushed cells (second condition), but only in the whole cells (first condition). In the supernatant (second condition), no glycosylated derivatives should be found since the enzyme con be present only in the cell and not directly in the supernatant.
3) Aglycone flavonoid can act as epigenetic probe but not as precursor. The epigenetic probe is capable of unlocking certain DNA reading areas for at least one metabolic pathway different from the second hypothesis. In this case, the aglycone flavonoid acts only as a probe, but not as a precursor since the metabolic pathway is not induced. As with the second hypothesis, the enzyme cannot be generated and/or mobilised outside of the cell, since DNA is not alive outside the cel. But, contrary to the second hypothesis, the aglycone flavonoid can only act as epigenetic probe, so other molecular families than aglycoslated derivatives will appear. Thus, in the supernatant (second condition), no glycosylated derivatives should be found either. With the whole cells (first condition), the aglycone flavonoid can be converted into a glycosylated derivative and the crushed calls (third conditioned trigger another chemical composition by opening other metabolic pathways.

### To Summaries:

the first hypothesis is evidenced if the whole calls (first condition) and the crushed calls third condition) convert the aglycone flavonoid into a glycosylated derivative.
the second hypothesis is evidenced if only the whole cells (first condition) convert the aglycone flavonoid into a glycosylated derivative.
the third condition is evidenced if the whole cells (first condition) convert the aglycone flavonoid into a glycosylated derivative and the crushed cells (third condition) trigger another chemical composition by opening other metabolic pathways.

### Protocol

Comparative tests were carried out on a pre-culture of Minard we obtained by in vitro cell culture according to the invention. Three conditions are tested:

For the first condition, the biomass consisting of suspended cells, partially lysed calls or whole cells, in the form of dusters or individuals and the culture medium (supernatant) are recovered and analysed separately.

For the second condition, the biomass is filtered to recover the cells and remove the supernatant. Only cells are analysed.

For the third condition, the biomass is filtered to recover the cells and remove the supernatant. These calls are transferred to new medium then crushed, for example by sonication and/or freezing.

For each condition, what is recovered is divided into two vials, with the contents of one vial then stimulated with naringenin and methyl jasmonate, with the contents of the other vial not being stimulated, thus consisting of the control.

The dosages of rosmarinic acid and naringenin derivatives are carried out on the contents of each sial according to the following:
For vials with stimulated contents: after recovery of the cells, supernatant, or crushed cells and before stimulation (T=0), 24 hours after stimulation (T=24 hours) and 48 hours later the first stimulation and just after a new stimulation (T=48 hours).
For vials without stimulated contents: after recovery of the cells, supernatant, or crushed cells (T=0), 24 hours later (T=24 hours) and 48 hours later (T=48 hours).

### Results

### Content of Rosmarinic Acid and Glycosylated Derivatives of Naringenin Under the Different Conditions:

TABLE 6

| | | First condition: | | Second condition: | | Third condition: | |
|---|---|---|---|---|---|---|---|
| | | cells | | supernatant | | crushed cells | |
| | | | Not | | Not | | Not |
| | | Stimulated | stimulated | Stimulated | stimulated | Stimulated | stimulated |
| T = 0 | Naringenin | / | 0 | / | 0 | / | 0 |
| | Glycosylated | / | 0 | / | 0 | / | 0 |
| | derivatives of | | | | | | |
| | naringenin | | | | | | |
| | Rosmarinic acid | / | + | / | 0 | / | 0 |
| T = 24 | Naringenin | 0 | 0 | +++ | 0 | +++ | 0 |
| hours | Glycosylated | + | 0 | 0 | 0 | 0 | 0 |
| | derivatives of | | | | | | |
| | naringenin | | | | | | |
| | Rosmarinic acid | +++ | ++ | 0 | 0 | 0 | 0 |

The results show that:
For the first condition, before stimulation, the cells contain a small quantity of rosmarinic acid. 24 hours alter stimulation with naringenin and methyl jasmonate, the stimulated cells contain glycosylated derivatives of naringenin and a greater amount of rosmarinic acid.
For the second condition, before stimulation, the supernatant does not contain rosmarinic acid. 24 hours after stimulation, the supernatant stimulated by naringenin and methyl jasmonate contains neither glycosylated derivatives of naringenin nor rosmarinic acid. However, the naringenin added during stimulation is still present in the supernatant 24 hours after stimulation, so it has not been degraded and/or transformed. This is explained by the fact that the enzyme capable of transforming naringenin into glycosylated derivatives of naringenin is not present in the supernatant.
For the third condition, before stimulation, the crushed cells do not contain rosmarinic acid. This is because rosmarinic acid is not a stable molecule outside the call if it is not protected by a solvent, it easily oxidizes and degrades. 24 hours after stimulation, the crushed cels stimulated by naringenin and methyl jasmonate contain neither glycosylated derivatives of naringenin nor rosmarinic acid, but only the naringenin added. This is because the enzyme capable of transforming naringenin into glycosylated naringenin derivatives is not present when the cellular material is outside the cell and the potentially produced rosmarinic acid is rapidly degraded.

To conclude, it has been demonstrated that according to the invention, the naringenin plays the role of an epigenetic probe in the cell. It sends signals to the DNA to generate and/or mobilize specific enzymes capables of transforming naringenin into glycosylated derivatives of naringenin.

### 5. In Vitro Efficacy Tests

A certain number of biological activity tests were carried out on the extracts according to the invention manufactured as described in point 2. above. They demonstrate many potential cosmetic activities.

A study of variances and a Student t-test for paired series are carried out for each test in order to judge the significance of the results.

### 5.1. From the Extract Monarda didyma

### 5.1.1. Skin Anti-Aging Activity

### 5.1.1.1. Protection Against Oxidative Stress

Oxidative stress plays a central role in the cutaneous response to various stresses. Free radicals (H₂O₂, OH—, O₂—) O₂, ¹O₂ . . . ) lead to protein, lipid, and DNA damages, causing premature aging of the skin and its appendages.

### Protocol

Normal human fibroblasts (HNF) are grown to confluence in their culture medium. The cells are then brought into contact with the composition according to the invention for 24 hours and then receive a fluorescent probe intended to mark the ROS intracellular production. After incorporation for 30 min and rinsing, the cells receive again the composition according to the invention and either nothing or an agent intended to create ROS (oxidative stress). The quantity of intracellular ROS is estimated by a fluorescence reading (e.g.: 490 nm/cm: 520 nm). The number of cells is estimated using the Hoechst 33258 method (DNA staining) to weight the data obtained. For melanocytes, an equivalent protocol was used.

### Results

Variation in the ROS production in fibroblasts (n=3) and melanocytes (n=5) with or without oxidative stress. Effect of 0.32% of the extract according to the invention compared to the control:

TABLE 7

| | Fibroblasts | | Melanocytes | |
|---|---|---|---|---|
| | Variation (%); | | Variation (%); | |
| | significance | | significance | |
| | Not stressed | Stressed | Not stressed | Stressed |
| Control | Reference 1 | Reference 2 | Reference 1 | Reference 2 |
| 0.32% of | −68%; | −94%; | −60%; | −80%; |
| the extract | p < 0.01 | p < 0.01 | p < 0.01 | p < 0.01 |
| according | | | | |
| to the | | | | |
| invention | | | | |

These results show that the extract according to the invention significatively reduces the intracellular content of ROS, either in the fibroblasts or the melanocytes, either having received oxidative stress or not.

Monarda didyma extract according to the invention therefore has a strong antioxidant capacity, making it possible to effectively fight against premature aging of the skin.

### 5.1.1.2. Protection of the Dermo-Epidermal Junction (DEJ)

DEJ assures the cohesion between the dermis and the epidermis. During aging, a decrease of synthesis of its components (especially collagen and laminin) is observed. The aging of the DEJ has significant repercussions on the resilience of the skin and the loss of its dynamism.

### 5.1.1.2.1. Stimulation of collagen-VII and laminin synthesis

### Protocol

Human keratinocytes (HK) were cultured at sub-confluence and were brought into contact or not (for the control cases) with the extract according to the invention. After this contact, the culture supernatants and the cell layers are measured respectively for their content of collagen VII and laminins are evaluated using ELISA type kits. The number of cols is estimated using the Hoechst method and used to standardize the results.

### Results

Variation of the collagen-VII (n=4) and laminins (n=4) production in keratinocytes. Effect of 0.66% of the extract according to the invention compared to the control:

TABLE 8

| | Collagen- | Variation | | Variation |
|---|---|---|---|---|
| | VII | (%); | Laminins | (%); |
| | (ng/10⁶ cell) | significance | (ng/10⁶ cell) | significance |
| Control | 372 ± 45 | Reference | 88.8 ± 7.2 | Reference |
| 0.66% of | 2503 ± 195 | +572%; | 128.8 ± 10.8 | +45%; |
| the extract | | p < 0.01 | | p < 0.01 |
| according to the | | | | |
| invention | | | | |

These results show that the extract according to the invention stimulates the production of collagen-VII and laminins, essential elements of the DEJ.

### 5.1.1.2.2. Stimulation of the synthesis of collagens-IV and -XVII

### Protocol

A gel comprising the extract according to the invention was applied daily during 7 days to the surface of skin explants. Then, the skins were cut and the collagen-IV and collagen-XVII synthesis were revealed by immunohistochemistry (marks of the sections with a first antibody specific for the protein to be assayed and revelation of this labelling by a fluorescent secondary antibody specific of the first antibody). Then, the intensity of the labelling was quantified by image analysis of the taken photographs.

Furthermore, the extract according to the invention at 0.5% or its placebo (control) was applied on human keratinocytes in culture and collagen VII synthesis was measured by ELISA on cellular extract after crushing. The results were reduced to the number of cells evaluated by counter labelling of the nuclei using the fluorescent dye Hoechst 33258, which marks the DNA.

### Results

Variation of collagen IV and collagen-XVII synthesis on skin explants after 7 days (n=3). Effect of 0.66% of the extract according to the invention compared to the control:

TABLE 9

| | Collagen- | Variation | Collagen- | Variation |
|---|---|---|---|---|
| | IV | (%) | XVII | (%); |
| | (AUF*) | significance | (AUF*) | significance |
| Control | 66.0 ± 2.6 | Reference | 9.7 ± 1.9 | Reference |
| 0.66% of the extract | 80.6 ± 11.2 | +22%; | 12.6 ± 1.4 | +30%; |
| according to the | | p < 0.01 | | p < 0.01 |
| invention | | | | |

*AUF: Arbitrary units of fluorescence

These results show that the extract according to the invention stimulates the production of collagen-IV and -XVII, which are also essential elements of the DEJ.

All these results show that the Manan's day extract according to the invention has a direct action to reinforce the JDE by stimulating the collagens and laminins constituting it. The extract according to the invention can ad on skin aging linked to a disorganization of the JDE, by counteracting the loss of suppleness and elasticity that it causes. In addition, collagens-IV and -XVII are known to be involved in the proper melanocyte anchoring. Stimulation of their synthesis by the extract according to the invention makes it possible to avoid premature aging of the melanocyte leading to the appearance of pigmentation defects and a loss of uniformity of the complexion.

### 5.1.1.3. Protection of the Dermal Extracellular Matrix

Elastase and MMPs (Matrix Metallo Proteases: dermal matrix proteases) are proteases that destroy extracellular matrix proteins such as elastin or different collagens. Their production increases with age and acute of chronic stress amplifies this phenomenon. Excessive production of MMPs leads to a reduction in the strength of the dermis through the loss of its density and its refinement. Too much elastase production loads to a loss of skin elasticity.

### Protocol

Fibroblasts are grown to confluence in their culture medium. The calls are exposed to solar irradiation, once a day for 4 days. At the end of the exposure, the calls are brought into contact with the extract according to the invention. The total RNAs are extracted to analyse elastase, MMP-2 and MMP-3 by RT-qPCR.

### Results

Variation of elastase (n=4) MMP2 (n=4) and MMP3 (n=3) quantity in irradiated fibroblasts. Effect of 0.16% of the extract according to the invention compared to the control:

TABLE 10

| | Elastase | MMP2 | MMP3 |
|---|---|---|---|
| Variation (%); significance | −33%; | −27%; | −30%; |
| | p < 0.05 | p < 0.05 | p < 0.05 |

The results show that elastase, MMP2 and MMP3 quantities are significantly reduced by the extract according to the invention. By limiting the production of proteases, the extract reduces their overall harmful activity on extracellular matrix (ECM) proteins, such as collagens and elastin, thus contributing to good health of the dermis and the protection of the microenvironment of the calls present.

### 5.1.1.4. Stimulation of the Molecule's Synthesis of Dermal Extracellular Matrix

### 5.1.1.4.1. Stimulation of Elastin Synthesis

Elastin is a protein of the dermal extracellular matric. The elasticity of the skin is modified during aging due to the decrease of elastin quantity produced leading to inappropriate assembly of elastin fibres.

### Protocol

Human dermal fibroblasts are grown to confluence in their culture medium. The calls are bathed in a buffer and exposed to UV once a day for 8 consecutive days at 30 mJ/cm² using the solar lamp model (UV Technology, Horle; 60 mJ/cm²). After each irradiation, the cells receive their culture medium containing the extract according to the invention. At the end of this contact, the cell layers are rinsed, fixed and marked with an anti-elastin antibody. The labelling was revealed using a fluorescent secondary antibody and photographs captured under a microscope. Image analysis on these photographs allowed elastin production to be quantified. A counter-labelling of the nuclei was performed using the fluorescent dye HOECHST 33258, which marks the DNA, to evaluate the cel population and thus to weight the fluorescence data obtained.

### Results

Variation of elastin production in fibroblasts exposed or not to sunlight (n=3). Effect of 0.16% of the extract according to the invention compared to the control:

TABLE 11

| | Non-irradiated | Irradiated fibroblasts |
|---|---|---|
| | fibroblasts | Reference |
| Control | Reference not exposed | UV exposed |
| Variation (%); significance | +196%; p < 0.01 | +524%; p < 0.01 |

The results show that the extract according to the invention significantly increase the elastin production in fibroblasts, either under stress conditions or not. In both cases, these increases are large and significant. This stimulation is very interesting because with age, elastin production decreases and this phenomenon is accentuated during exposure to the sun.

### 5.1.1.4.2. Stimulation of Collagen-I Synthesis

Collagen I is the most abundant protein of the dermis. With age, dermal fibroblasts produce fewer supporting proteins, including collagen I. Therefore, it is essential, in particular to have beautiful and firm skin.

### Protocol

The same protocol that was used to that described in point 6.1.1.2.2 above.

### Results

Variation of collagen-I quantity on the skin explants after 7 days (n=4). Effect of 0.66% of the extract according to the invention compared to the control:

TABLE 12

| | Collagen-I | Variation (%); |
|---|---|---|
| | (AUF) | significance |
| Control | 20.8 ± 3.3 | Reference |
| 0.66% of the extract according | 35.5 ± 7.1 | +71%; p < 0.01 |
| to the invention | | |

The results show that the extract according to the invention clearly and significantly stimulates the collagen-I production.

The elastin and collagen I production ensures the good mechanical properties of the skin, firmness, resilience and elasticity, and thus, for example, prevents and/or treats skin sagging, wrinkles and fine lines.

### 5.1.1.5. Protection Against Glycation

Protein glycation by reducing sugars of the skin is responsible of premature aging of the skin.

Enzymatic and/or structural properties of proteins once glycated are altered, which disrupts the proper functioning of the involved cells or organisms. This alters the mechanical and elastic properties of the dermal, which becomes less flexible, more rigid, but also more flabby and less reactive. This also results in a dull complexion.

### Protocol

This test uses a model protein, the serum albumin, which serves as a target, and an edible reducing sugar from fruits. The protein is gradually glycated (bound to sugar) in an irreversible manner, in the presence or absence of the extract according to the invention. The glycation changes are monitored by fluorescence.

### Results

Variation of glycation (n=2). Effect of 0.66% of the extract according to the invention compared to the control

TABLE 13

| | Variation (%); significance |
|---|---|
| Control | Reference |
| 0.66% of the extract according to | −91.9%; p < 0.05 |
| the invention | |

These results show the strong anti glycant potential of the active agent according to the invention which can helps fight against skin aging and loss of radiance of the complexion.

### 5.1.1.6. Anti-Spot Activity

The melanocyte is the pigment call of the skin. Keeping the melanocyte alive and healthy ensures quality pigmentation and limits the formation of black and white spots which are common during skin aging.

### 5.1.1.6.1. Protection of Melanocyte Dendrites

The melanocyte produces melanin abundantly and distributes it to neighbouring keratinocytes through branched extensions called dendrites. The keratinocytes absorb melanin, helping to protect the cell and its DNA from the effects of the sun. During aging, dendrites are smaller, and melanocytes are larger. These dendrites accumulate melanin without being able to distribute it, which poisons them.

### Protocol

Human melanocytes in culture and at low density are brought into contact with the extract according to the invention and are stressed by H₂O₂ which is a molecule strongly produced under UV stress. The length of the melanocyte dendrites is quantified by image analysis.

### Results

Variation of the length of the melanocyte dendrites stressed by H₂O₂ (n=4). Effect of 0.50% of the extract according to the invention compared to the control case:

TABLE 14

| | Dendrites | Dendites | Variation |
|---|---|---|---|
| | lengths | lengths 2 hours | (%); |
| | at T0 (μm) | after stressed (μm) | significance |
| Control | 70.5 ± 7.6 | 54.8 ± 6.8 | −22.0%; |
| | | | p < 0.05 |
| 0.50% of the extract | 67.2 ± 1.1 | 62.0 ± 2.4 | −8.0%; |
| according to | | | p < 0.01 |
| the invention | | | |

The results show that the extract according to the invention strongly protects the melanocytes from stress induced by H₂O₂ compared to the control case (placebo) since the retraction of the dendrites is much less significant.

### 5.1.1.6.2. Increase in the Number of Young Melanocytes

The presence of spindle-shaped melanocytes is a good criterion for the youthful state of the population of cells observed. When the population is older, the melanocytes are larger.

### Protocol

Human melanocytes are pushed into premature senescence. Then, they are cultured in the presence of the extract according to the invention for 19 consecutive days, thereby showing its safety for these cels, which are nevertheless considered very fragile. Cel media being replaced every 2 to 3 days. Cells were then photographed and those a count of spindle-shaped cells were quantified and compared to the total number of cells.

### Results

Variation of the spindle cells number in melanocyte cultures (n=3). Effect of 0.16% of the extract according to the invention compared to the control:

TABLE 15

| | Spindle cells (% vs | Variation (%); |
|---|---|---|
| | total cells) | significance |
| Control | 35 ± 12 | Reference |
| 0.16% of the extract | 67 ± 13 | +91%; p < 0.01 |
| according to | | |
| the invention | | |

The results shows that the extract according to the invention significantly increases the number of spindle calls in the total population of melanocytes, which means that the population of melanocytes is less senescent and therefore younger.

### 5.1.1.6.3. Increased Hyaluronic Acid Synthesis by Melanocytes

Hyaluronic acid is produced by fibroblasts, by keratinocytes and also by melanocytes. Hyaluronic acid interacts, via receptors, with keratinocytes and melanocytes in the epidermis.

### Protocol

Human melanocytes in culture and joined receive the extract according to the invention for 24 hours. After rinsing, cells are UVB exposed in a buffer before being cultured in a medium containing the extract according to the invention. The calls are then peeled off and extracted from the medium. Hyaluronidases are deactivated by heating and the pericellular hyaluronic acid content is estimated by an ELISA method. The number of cels is estimated using the Hoechst method and used to standardize the results.

### Results

Variation of hyaluronic acid quantity around melanocyte after UVB exposed (n=4). Effect of 0.32% of the extract according to the invention.

TABLE 16

| | Hyaluronic acid | Variation (%); |
|---|---|---|
| | (ng/10⁶ cell) | significance |
| Control, UVB stressed | 594 ± 125 | Reference |
| 0.32% of the extract according | 763 ± 102 | +28%; p < 0.08 |
| to the invention | | |

The results show that the extract according to the invention can significantly increase the hyaluronic acid production around the melanocytes when the cells are irradiated. This is of great interest in preventing the emergence of melanocyte senescence.

### 5.1.1.6.4. Reduction of Senescent Enzyme Activity

Enzyme SA bata-galactosidase (senescence-associated beta-galactosidase) is very active in senescent cells while it is not or only slightly active in young cells.

### Protocol

Human melanocytes are pushed into premature senescence. Then, they are cultured with the extract according to the invention during 5 consecutive days. Fresh medium is added on the third day to maintain call survival. Cells are labelled with an indole galactose derivative allowing to quantify the SA-bata-galactosidase enzymatic activity whose accumulation in the lysosomes of senescent cells appears in blue.

### Results

Variation of SA-beta-galactosidase enzyme in the melanocyte culture (n=5). Effect of 0.16% of the extract according to the invention compared to the control:

TABLE 17

| | SA beta-galactosidase activity | Variation (%); |
|---|---|---|
| | (% labelled vs total cells) | significance |
| Control | 60 ± 13 | Reference |
| 0.16% of the extract | 38 ± 13 | −37%; |
| according to | | p < 0.01 |
| the invention | | |

The results show that the extract according to the invention significantly reduces the activity of the SA beta-galactosidase enzyme which accumulates in senescent cels. The extract according to the invention therefore can be reduce the number of senescent calls.

### 5.1.1.6.5. Reduction of the DKK-1 Production

Excess production of the DKK1 protein in the dermis leads to melanocyte senescence and therefore to the total cessation of melanin production.

### Protocol

Fibroblasts are grown to confluence in their culture medium. The cells are then brought into contact with the extract according to the invention. At the end of this contact, fibroblasts are exposed to UVB stressed, then they are brought again into contact with the extract according to the invention. Culture supernatants are assayed for the DKK-1 content, a molecule identified as pro-senescent for melanocytes.

The quantity of cells is estimated using the Hoechst method and used to standardize the results.

### Results

Variation of DDK-1 quantity in UVB stressed fibroblasts (n=3). Effect of 0.66% of the extract according to the invention compared to control:

TABLE 18

| | DKK-1 | Variation (%); |
|---|---|---|
| | (ng/10⁵ cell.) | significance |
| UVB stressed control | 16457 ± 1980 | Reference |
| 0.66% of the extract | 7030 ± 591 | −57%; p < 0.01 |
| according to the | | |
| invention | | |

The results show that the extract according to the invention significantly reduces the DKK-1 production in fibroblasts. This reinforces the interest of the extract according to the invention for reducing the progression of formation of the senescent phenotype in melanocytes.

### 5.1.1.6.6. Reduction of the Inflammation Markers Production

IL-6 (interleukine-6) and IL-8 (interleukin-8) are part of the secretions increased by senescent cells. In addition, IL-6 is known for its pro-inflammatory aspects and as an actor in DNA breaks and IL-0 is a molecule calling and exciting macrophages which thus release of other cytokines and matrix proteases.

### Protocol

Melanocytes are UVB irradiated once a day for two days to induce their senescence. The cells are then brought into contact with the extract according to the invention between each radiation, i.e., for a total of 48 hours. Then, dosage of IL-6 and IL-8 production in the culture medium was carried out using the Hoechst method and used to standardize the results.

### Results

Variation of IL-6 (n=6) and IL-8 (n=6) quantities in melanocytes, after UVB irradiation. Effect of 0.329% of the extract according to the invention compared to control:

TABLE 19

| | IL-6 | Variation (%); | IL-9 | Variation (%); |
|---|---|---|---|---|
| | (pg/10 [symbol] cell) | significance | (ng/10 [symbol] cell) | significance |
| UVB irradiated control | 253.8 ± 14.0 | Reference | 793.5 ± 38.4 | Reference |
| 0.32% of the extract | 137.0 ± 12.3 | −46%; p < 0.01 | 215.9 ± 13.4 | −73%; p < 0.01 |
| according to the invention | | | | |

[symbol]

indicates data missing or illegible when filed

The results show that the extract according to the invention significantly reduces the IL-6 and IL-9 quantities in melanocytes.

### 5.1.1.6.7. Conclusion

All of these results in points 6.1.1.6.1 to 6.1.1.6.6 above show that the extract according to the invention can be to slow down the senescence of melanocytes, and consequently to show down the appearance of pigmentary disorders such as white and or black senescence spots.

### 5.1.2. Moisturizing Activity and Epidermis Protection Against External Aggressions

### 5.1.2.1. Stimulation of Hyaluronic Acid Synthesis

Hyaluronic acid is a major constituent of the epidermis contributing to the barrier function as well as to the maintaining of a satisfactory hydration of the skin. It is capable of absorbing 1,000 times its own weight in water. It is in the form of an aqueous and nourishing gal which files the spaces between the keratinocytes. It prevents from dryness of the skin, which is known to alter the texture of the skin, giving it a rough touch.

### Protocol

Human keratinocytes are cultured at sub-confluence and then contacted or not (for the control cases) with the extract according to the invention. After this contact, the synthesis of hyaluronic acid is assayed using ELISA methods. The cell concentration is estimated using the Hoechst method.

### Results

Variation of hyaluronic acid production in keratinocytes (n=4). Effect of 0.32% of the extract according to the invention compared to control:

TABLE 20

| | Hyaluronic | |
|---|---|---|
| | acid | Variation (%); |
| | (ng/10 [symbol] cell) | significance |
| Control | 2255 ± 151 | Reference |
| 0.32% of the extract | 7631 ± 469 | +238%; p < 0.01 |
| according to the | | |
| invention | | |

[symbol]

indicates data missing or illegible when filed

These results show that the extract according to the invention significantly increases hyaluronic acid synthesis by keratinocytes.

### 5.1.2.2. Strengthening the Skin Barrier

Keratinocytes migrate from the basal layer, the deeper layer of epidermis, to the stratum corneum the most superficial layer, differentiating themselves into corneocytes. The stratum corneum is a semi permeable protective layer that prevents water loss and maintains skin hydration. A better differentiation of keratinocytes leads to a strengthening of the skin barrier and therefore contributes to better protection of the epidermis against external aggressions and to maintain a better hydration.

### Protocol

Human keratinocytes are cultured at sub-confluence and then contacted or not (for the control cases) with the extract according to the invention. Differentiation is monitored visually by observing the phenotype of the keratinocytes over 4 days.

### Results

TABLE 21

| | Pro-diferentiation |
|---|---|
| | effect observed |
| Condtion | after 4 days |
| Control | 0 |
| 0.66% of the extract | ++ |
| according to the invention | |

The assessment is visually. In the control case, the untreated cels show a carpet of contiguous keratinocytes with well-defined cellular contours. On the other hand, in the presence of the extract according to the invention the cells retract and the contacts between the cells decrease until there are empty spaces between the cells, which represents a characteristic aspect of advanced differentiation of keratinocytes;

These results demonstrate that Monarda didyma extract contributes to better protection of the epidermis and better hydration of the skin.

### 5.1.3. Anti-Seborrheic Activity

Oly sch is associated with excessive sebum production by the sebocytes. Too much sebum leads to changes in the properties of the skin and scalp, for example by increasing the formation of pimples and blackheads and dogging pores, which then become dilated and more visible, leading to uneven skin texture and/or excessive growth of the bacteria responsible for dandruff, such as Malassezia fungi on the scalp.

An anti-seborrheic cosmetic active ingredient will counteract this development by reducing sebum production, which will have the effect of tightening skin pores, smoothing it and reducing the oily/shiny appearance with irregular texture characteristic of oily skin and/or making the scalp heathier, with less dandruff and associated itching.

### Protocol

Sebocytes am seeded in their growth medium. At confluence, the cells are contacted or not (control case) with the extract according to the invention for 48 hours. After removing media, monolayers are incubated with Nile Red marker of intracellular lipids which estimates the amount of lipids in the cells. The estimate of the viability is performed in parallel on the same layers using a fluorescent dye.

### Results

Variation of lipidic synthesis by sebocytes (n=3). Effect 0.66% of the extract according to the invention compared to control:

TABLE 22

| | Variation (%); |
|---|---|
| | significance |
| Control | Reference |
| 0.66% of the extract | −61%; p < 0.01 |
| according to the invention | |

These results show that sebocytes exposing to the extract according to the invention reduces the quantity of lipids in sebum-producing cells.

The extract according to the invention can be used to treat skin disorders associated with oily skin.

### 5.1.4. Slimming Activity

### 5.1.4.1. Lipolytic Effect

Glycerol is a product of the triglyceride's hydrolysis. Stimulating its production in the presence of an active ingredient therefore reflects an increase in lipolysis. Increasing lipolysis will therefore reduce the size of adipocytes and therefore reduce the adipose tissue.

### Protocol

Human pre-adipocytes are seeded and induced to differentiate with a specific cocktail of inducers. To obtain mature adipocytes well loaded with triglycerides, the cells are brought into contact with the extract according to the invention in a maintenance culture medium.

The supernatants are then recovered. The quantity of glycerol released from the hydrolysis of the intracellular triglycerides is measured every day for 4 days using a commercial kit from Sigma.

In parallel, a survival assay is carried out by Hoechst staining to quantify the number of cels.

Visual inspection is carried out before each recovery to verify the absence of toxicity.

### Results

Variation of the concentration of glycerol released by adipocytes after 4 days of contact with the extract according to the invention (n=3). Effect 0.66% of the extract according to the invention compared to control:

TABLE 23

| | Variation (%); |
|---|---|
| | significance |
| Control | Reference |
| 0.66% of the extract | +152%; p < 0.01 |
| according to the invention | |

These results show that the extract according to the invention increases the lipolysis of the adipocytes.

### 5.1.4.2. Anti-Lipogenesis Effect

Glycerol-3-Phosphate Dehydrogenase (G3PDH) is a fat storage enzyme whose expression increases strongly during the differentiation of pre-adipocyte fibroblasts into adipocytes. As inhibition sill reduce therefore the quantity of adipocytes and therefore the adipose tissue.

### Protocol

Mature human adipocytes are formed from an immature state using a hormonal cocktail. A series receives the extract according to the invention during this phase. The increase in adipose reserves is compared to control casas visually and by measuring the activity of the fat storage enzyme (G3PDH). A viability test is carried out in parallel.

### Results

Variation of G3PDH activity in adipocytes in the way of differentiating (n=3). Effect 0.66% of the extract according to the invention compared to control:

TABLE 24

| | Variation (%); |
|---|---|
| | significance |
| Control | Reference |
| 0.66% of the extract | −80%; p < 0.01 |
| according to the invention | |

These results show that the extract according to the invention slows down the differentiation of the adipocytes significantly.

In conclusion, advantageously, the extract according to the invention is doubly effective, on the one hand to increase lipolysis and, on the other hand, to slow down lipogenesis. Therefore, the extract has strong slimming power.

### 5.1.5. Soothing Activity of the Skin

Inflammation mediators, such as IL-6 and PGE₂ are very present in microinflammatory phenomena. Reducing the presence of these mediators has the effect of reducing feelings of discomfort in sensitive and reactive skin.

### Protocol

Normal human dermal fibroblasts are grown in their culture medium until a confluent mat is obtained. The calls are then brought into contact with the extract according to the invention for 24 hours and then the cells mat is UVB irradiated and brought again into contact with the extract according to the invention for 24 hours. The quantities of PGE; and IL-6 synthesized are measured in the culture supernatants by ELISA assay.

The number of cells is evaluated to weight the data obtained.

### Results

Variation of IL-6 and PGE₂ quantities in UVB irradiated fibroblasts (n=3). Effect 0.66% of the extract according to the invention compared to control:

TABLE 25

| | IL-6 | PGE₂ |
|---|---|---|
| | (pg/10 [symbol] cell) | (pg/10 [symbol] cell) |
| Variation (%) compared | −82%; p < 0.01 | −81%; p < 0.01 |
| to control; significance | | |

[symbol]

indicates data missing or illegible when filed

Advantageously the extract according to the invention strongly and significantly reduces the two pro-inflammatory messengers tested.

Therefore, the extract according to the invention can be used to soothe skin discomfort in sensitive skin such as redness, tightness, etc.

### 5.1.6. Conclusion

All of in vitro results presented above show that the Monarda didyma extract prepared according to the process of the invention advantageously possesses a very wide range of biological activities having great interest, particularly in cosmetics.

Monarda didyma extract according to the invention limits the reduction in the youthfulness of calls by limiting the impact of oxidative stress induced from the outside. The microenvironment of the calls is also protected, by limiting the production of proteases (elastases and MMPs) but also by promoting the production of the elements of the JDE which are collagens-IV, -VII, -XVII, hyaluronic acid, and laminins. Furthermore, Monarda didyma extract according to the invention acts by stimulating the production of elastin and collagen-I, elements of the dermal matrix which are known to be strongly impacted by natural aging. This makes it possible to maintain and strengthen the microenvironment of the cells close to the DEJ and avoids deregulation following the weakening of the latter. Finaly, the Monad w extract according to the invention is favourable for keeping the melanocyte alive and in good health, making it possible to guarantee quality pigmentation and thus to limit the formation of black and white spots which are common during the aging of the skin.

### 5.2. From the Extract Lavandula angustifolia

The testing protocols are identical to those described for Monarda didyma in point 6.1 above.

### 5.2.1. Anti-Aging Activity of the Skin

### 5.2.1.1. Protection Against Oxidative Stress

### Results

Variation of ROS production in fibroblasts (n=3) with or without oxidative stress. Effect of 0.05% of the extract according to the invention compared to the control:

TABLE 26

| | Fibroblasts | Fibroblasts |
|---|---|---|
| | without | with |
| | oxidative | oxidative |
| | stress | stress |
| Control | Reference 1 | Reference 2 |
| Variation (%); significance | −45%; p < 0.01 | −53%; p < 0.01 |

The results show that the extract according to the invention can be significantly reduce the intracellular content of reactive oxygen species in fibroblasts having received oxidative stress or not.

Therefore, Lavandula angustifolia extract according to the invention has a strong antioxidant capacity, making it possible to effectively light against premature aging of the skin.

### 5.2.1.2. Protection Against Glycation

### Results

Variation of glycation. Effect of 0.05% of the extract according to the invention compared to the control:

TABLE 27

| | Variation (%); |
|---|---|
| | significance |
| Control | Reference |
| 0.05% of the extract | −96%; p < 0.01 |
| according to the invention | |

These results show the strong anti-glycant potential of the active agent according to the invention which can helps fight against skin aging and loss of radiance of the complexion.

### 5.2.2. Moisturizing Activity

### 5.2.2.1. Hyaluronic Acid

### Results

Variation of hyaluronic acid production in keratinocytes (n=5). Effect of 0.01% of the extract according to the invention compared to control:

TABLE 28

| | Variation (%); |
|---|---|
| | significance |
| Control | Reference |
| 0.01% of the extract | +185%; p < 0.01 |
| according to the invention | |

These results show that the extract according to the invention significantly increases hyaluronic acid synthesis by keratinocytes.

### 5.2.3. Soothing Activity of the Skin

### 5.2.3.1. Pro-Inflammatory Messengers

### Results

Variation of PGE₂, IL-6 and IL-8 production in UVB-irradiated fibroblasts. Effect of 0.0125% of the extract according to the invention compared to the control:

TABLE 29

| | IL-6 | IL-8 | PGE₂ |
|---|---|---|---|
| | (pg/10⁶ cell) | (pg/10⁶ cell) | (pg/10⁶ cell) |
| Control | Reference | Reference | Reference |
| Variation (%); | −96%; p < 0.01 | −59%; p < 0.01 | −97%; p < 0.01 |
| significance | | | |

Advantageously the extract according to the invention strongly and significantly reduces the three pro-inflammatory messengers tested.

### 5.2.3.2. Inhibition of Cannabinoid 2 Receptor (CB2)

The cannabinoid receptor 2 (CB2) is expressed on the skin and its activation is associated with anti-inflammatory, arti-antioxidant, seboregulatory and immunomodulatory effects, but without psychoactive effects.

### Protocol

An agonist with specific affinity for CB2 is radio labelled. The extract according to the invention is placed with this radio-labelled agonist in the presence of a membrane containing the CB2 receptor. The binding of said extract to the CB2 receptor is evaluated by comparing the radioactivity (binding of the agonist) of a control case compared to the condition containing said extract.

### Results

Variation in inhibition of CB2 receptor binding. Effect of 0.05% of the extract according to the invention compared to the control:

TABLE 30

| | % of binding inhibition |
|---|---|
| | CB2 |
| Control | Reference |
| Variation (%) | 49.4% |

The results show that the extract according to the invention binds to the CB2 receptor instead of the specific control.

Therefore, the extract according to the invention can be used to soothe skin discomfort in sensitive skin such as redness, tightness, etc.

### 6. In Vivo Efficacy Tests

The tests carried out relate as an example, to the original cosmetic activity of the Monarda didyma extract prepared according to the process of the invention by in vitro cell culture to treat senescence spots.

### 6.1. Principle and Protocol of In Vivo Tests

Skin spots, sometimes brown and sometimes white, are one of the first visible signs of skin aging. The origin of these spots, as explained above, lies in the pigment cell of the skin: the melanocyte.

Brown spots are caused by an overproduction of melanin.

Conversely, white spots are due to an absence of melanin. In addition, these spots have degraded matrices in the dermis and DEJ and their surface is flat, which makes the skin less flexible and more rigid.

### Product Tested

The cream described in point 4, above.

### Protocol

The evaluation of the effectiveness of the cream was carried out for 56 days on a total of 62 volunteers in two studies carried out against placebo. These studies enabled to evaluate the effect of the cream on the various types of brown and white spots present on the skin of volunteers.

### Specific Inclusion Criteria

The first study was carried out on a first panel of 27 women (average age 58 years [49-67]) including a test using a multispectral camera and a test using a photographic bench.

The second study was carried out on a second panel of 25 women (average age 59 years [47-69]) including a test using a Cutometer®.

The following criteria also had to be respected for the two recruited panels: being menopausal or peri-menopausal, phototype II to IV and having imperfect skin, i.e., having visible brown spots on the face and arms and visible white spots on the arms.

### Type of Study, Duration, Applications

For 2 months, the volunteers applied to the face and forearms, twice a day, a cam according to the invention and a placebo cream contralaterally.

### Statistics

For the quantification of the spots, statistical studies were performed using the Student t-test or, if necessary with a non parametric Wilcoxon lost on paired series.

In the case of expert evaluations, Khi2 tests were used to compare the frequencies of responses.

### 6.2. Evaluation of Brown and White Spots by Multispectral Camera

### Protocol

An Antera 3D® Multispectral LED camera was used, providing three dimensions images at different wavelengths exposure of visible light. Facial photographs were taken in cross-polarized mode to eliminate any parasitic shine and increase the sharpness of spots.

This system allows several analyses in parallel and therefore provides several parameters:
colour parameters L′, a′, and b″ (CIELAB colour space);
relative quantities of chromophores such as haemoglobin and melanin
different topographical parameters such as wrinkles, texture or atrophic and hypertrophic volumes.

For brown spots, maximum pigmentation was studied and pigmentation heterogeneity which quantifies pigmentation variations within a spot was monitored.

For white spots, their volume and maximum depth was studied. In fact the white spots form a slight hollow whose smoothly surface, devoid of the microdepression network. These characteristics are linked to the breakdown of the underlying skin.

Among the panelists in the first panel above only 23 women had white spots with these characteristics, i.e., a depression in the skin and a sufficiently clear flattening.

### Results

Variation of maximum pigmentation and heterogeneity of skin brown spots. Effect of the cream according to the insertion:

TABLE 31

| | Maximum pigmentation | | Heterogeneity | |
|---|---|---|---|---|
| | Cream according | Placebo | Cream according | Placebo |
| | to the invention | cream | to the invention | cream |
| % of variation T56 days vs. T0; | −2.1%; p < 0.01 | −0.9%; p < 0.01 | −4.2%; p < 0.01 | −1.1%; p < 0.05 |
| significance vs T0 | | | | |
| % of improvement of the cream | −1.2%; p < 0.01 | | −3.3%; p < 0.05 | |
| according to the invention vs. | | | | |
| placebo cream | | | | |

Variation in maximum depth and volume of white skin spots. Effect of the cream according to the invention:

TABLE 32

| | Maximum depth | | Volume | |
|---|---|---|---|---|
| | Cream according | Placebo | Cream according | Placebo |
| | to the invention | cream | to the invention | cream |
| % of variation T56 days vs T0; | −23.2%; p < 0.01 | −9.5%; p < 0.01 | −49.6%; p < 0.01 | −18.1%; p < 0.65 |
| significance vs. T0 | | | | |
| % of improvement of the cream | −13.7%; p < 0.05 | | −31.5%; p < 0.05 | |
| according to the invention vs. | | | | |
| placebo cream | | | | |

The two tables result show that the cream according to the invention significantly reduces the pigmentation and heterogeneity of brown spots, as well as the depression created by white spots, making these two types of spots less visible and making it possible to have a more even complexion.

### 6.3. Evaluation of Brown Spots by Photographic Bench

### Protocol

A HeadScan® V05 photographic bench (Orion Concept, France) was used for volunteer's face photographs were taken in cross-polarized mode to eliminate any parasitic shine. A panel of 6 expert judges evaluated these photos on the criterion: the skin is more even and less stained.

### Results

Results showed 64% favourable responses for the cream according to the invention, which is significantly higher than 35% for the placebo cream.

### 6.4. Evaluation of Viscoelastic Parameters of White Spots

### Protocol

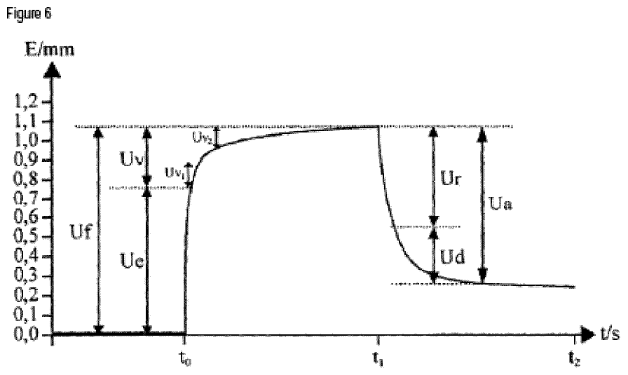

The Cutometer® (Courage & Khazaka) is used to measure the viscoelastic parameters of the skin. This device measures the deformation of a skin area, subjected to repeated mechanical suction stresses, as well as its recovery power. The device provides graphs of skin deformation as a function of time as shown in FIG. 6. In this FIG. 6, the parameters Uf, Ue and Ua are indicated, representing respectively the total elongation, the immediate elongation and elongation recovered after stress.

The measurements were carried out on the areas with the white spots. Three independent acquisitions were carried out at T0 and T=56 days.

### Results

Variation of total elongation (Un), immediate elongation (Ue) and recovered elongation (Ua) of cutaneous white spots (N=25). Effect of the cream according to the invention:

TABLE 33

| | Uf (total | | Ue (immediate | | Ua (recovered | |
|---|---|---|---|---|---|---|
| | elongation) | | elongation) | | elongation) | |
| | Cream | | Cream | | Cream | |
| | according | | according | | according | |
| | to the | Placebo | to the | Placebo | to the | Placebo |
| | invention | cream | invention | cream | invention | cream |
| % of variation T56 days | 10.5%; | 4.8%; | 12.4%; | 7.0%; | 13.3%; | 4.1%; nsd |
| vs. T0; significance vs. | p < 0.01 | p < 0.05 | p < 0.01 | p < 0.05 | p < 0.01 | |
| T0 | | | | | | |

| % of improvement of the | 5.7%; p < 0.05 | 5.4%; p < 0.01 | 9.2%; p < 0.05 |
|---|---|---|---|
| cream according to the | | | |
| invention vs. placebo | | | |
| cream | | | |

* nsd: non-significant data

The results show that the three parameters are batter after 56 days of application of the cream containing the extract according to the invention.

With the cream containing the extract according to the invention, the Uf increase associated with the Ue increase shows greater elongation of the skin which leads to skin that is less hard, more deformable, more flexible at the level of the spots white.

The concomitant increase of Ua shows that if the skin is more deformed, it also returns better to its original state, which proves good elasticity.

Therefore, the results show a softening of the hypopigmented areas as well as a better return after 56 days of application of the cream containing the extract according to the invention.

The cream according to the invention can improve the skin quality in terms of white spots which will be less visible.

### 6.5. Conclusion

All the in vivo results presented shove show that the active ingredient of Monarda didyma according to the invention advantageously can be to treat senescence spots. In fact, the active ingredient according to the invention acts on both types of spots, which are lass visible and allows to have a more homogeneous complexion.

1. Method for obtaining an extract of plant origin by in vitro plant culture, comprising, from a line of undifferentiated or dedifferentiated plant cells, the following steps successively:
a pre-culture step, intended to amplify the biomass of said plant cells;
a culture step of the biomass in a bioreactor comprising at least one proliferation phase, and
a treatment step of the harvested biomass to produce said extract comprising secondary metabolites of interest,
wherein at the culture step in the bioreactor at least one aglycone flavonoid is added to the culture medium.

2. Method according to claim 1, wherein the aglycone flavonoid is selected from aglycone flavanones and/or aglycone flavones.

3. Method according to claim 2, wherein the aglycone flavanones are selected from naringenin, eriodictyol, butine or a mixture thereof.

4. Method according to claim 2, wherein the aglycone flavones are selected from luteolin and apigenin or a mixture thereof.

5. Method according to claim 1, wherein the addition of the said at least one aglycone flavonoid is carried out during the exponential phase of proliferation of the biomass.

6. Method according to claim 1, wherein an elicitation is initiated during the step of culture in the bioreactor.

7. Method according to claim 6, wherein the elicitation is chemical, using an elicitor of biological origin selected from chitosan, methyl jasmonate, jasmonic acid and salicylic acid.

8. Method according to claim 1, wherein the treatment step comprises the removal of the culture medium.

9. Method according to claim 1, wherein the biomass treatment comprises a step of releasing the intracellular content outside the plant cells.

10. (canceled)

11. Method according to claim 9, wherein the treatment step comprises the elimination of cellular debris after the release of the intracellular content.

12. (canceled)

13. Method according to claim 1, wherein it is applied to plants of the Lamiaceae family.

14. Method according to claim 13, wherein the plant is selected from the Monarda and/or Lavandula genus.

15. Method according to claim 14, wherein the plant is Monarda didyma and/or Lavandula angustifolia.

16. (canceled)

17. (canceled)

18. (canceled)

19. Extract of plant origin obtained by the method according to claim 1.

20. Extract of plant origin according to claim 19, wherein it is obtained from plants of the Lamiaceae family and wherein it comprises glycosylated flavonoids and rosmarinic acid as secondary metabolites.

21. Extract according to claim 20 obtained from Monarda didyma, comprising glycosylated derivatives of naringinin as secondary metabolites.

22. Extract according to claim 20 obtained from Lavandula angustifolia, comprising glycosylated derivatives of luteolin as secondary metabolites.

23. Extract of plant origin according to claim 19, wherein it is obtained from plants of the Nyctaginaceae family and wherein it comprises glycosylated flavonoids.

24. Extract according to claim 23 obtained from Abronia villosa, comprising glycosylated derivatives of luteolin as secondary metabolites.

25. Cosmetic composition comprising an extract according to the claim 19 as active ingredient and a physiologically acceptable medium.

26. (canceled)

27. (canceled)

28. (canceled)

29. (canceled)

30. (canceled)
